(19) United States
(12) Patent Application Publication
(10) Pub. No.: US 2016/0067190 A1
(43) Pub. Date: Mar. 10, 2016

(54) INJECTABLE NANO-NETWORK GELS FOR DIABETES TREATMENT

(71) Applicants: THE CHILDREN'S MEDICAL CENTER CORPORATION, Boston, MA (US); MASSACHUSETTS INSTITUTE OF TECHNOLOGY, Cambridge, MA (US)

(72) Inventors: Daniel G. Anderson, Sudbury, MA (US); Zhen Gu, Cambridge, MA (US); Alex Arthur Aimetti, Waltham, MA (US); Robert S. Langer, Newton, MA (US)

(73) Assignee: The Children's Medical Center Corporation, Boston, MA (US)

(21) Appl. No.: 14/787,652

(22) PCT Filed: Apr. 29, 2014
(86) PCT No.: PCT/US2014/035927

§ 371 Date: Oct. 28, 2015

Related U.S. Application Data
(60) Provisional application No. 61/817,752, filed on Apr. 30, 2013.
(60) Provisional application No. 61/864,069, filed on Aug. 9, 2013.

(51) Int. Cl. A61K 9/51; A61K 38/44; A61K 9/00; A61K 38/28

(52) U.S. Cl. CPC A61K 9/5161; A61K 38/28; A61K 38/443; A61K 9/0019

(57) ABSTRACT

A system for “smart” delivery of a therapeutic, prophylactic or diagnostic agent, such as glucose-mediated delivery of insulin through an injectable nano-network consisting of oppositely-charged dextran nanoparticles encapsulating insulin and glucose-specific enzymes forming a gel-like 3D scaffold. As demonstrated by the examples, the system effectively dissociates to release insulin in a hyperglycemic condition, where the catalytic conversion of glucose into gluconic acid and the subsequent degradation of polymeric matrix are facilitated. This formulation design provides a delivery strategy for both self-regulated and long-term diabetes management.

### CROSS-REFERENCE TO RELATED APPLICATIONS

This application claims benefit of U.S. Provisional Application No. 61/817,752, filed Apr. 30, 2013, and U.S. Provisional Application No. 61/864,069, filed Aug. 9, 2013. Application No. 61/817,752, filed Apr. 30, 2013, and Application No. 61/864,069, filed Aug. 9, 2013, are hereby incorporated herein by reference in their entirety.

### FIELD OF THE INVENTION

This invention generally relates to smart or interactive delivery systems for therapeutics, prophylactic or diagnostic agents in response to glucose levels.

### BACKGROUND OF THE INVENTION

Diabetes mellitus is a disorder of glucose regulation with accumulation of glucose in the blood. In normal individuals, insulin is secreted basally, usually in the range of 0.5 to 1.0 units per hour, and the levels are increased after a meal. Responsive to the rise in blood glucose levels following a meal, the pancreas secretes a bolus of insulin, which returns blood glucose to normal levels by stimulating the uptake of glucose into cells and signaling the liver to reduce glucose production. There are normally two phases of insulin release in response to a meal. The early phase (responsible for shutting down hepatic glucose production) is a spike of insulin release that occurs within 2-15 minutes of eating. The late phase release extends about 2 hours. Between meals the liver breaks down glycogen stores to provide glucose to the brain and other tissues.

Diabetes results in chronic hyperglycemia due to the inability or reduced ability of the pancreas to produce adequate amounts of insulin or due to the inability or reduced ability of cells to synthesize and/or release insulin. In diabetics, the effectiveness of the first-phase response is decreased or absent, leading to elevated postprandial glucose levels. Diabetes is a major public health problem affecting 285 million people across the world and this number is expected to be over 450 million by 2030 (Wild, et al., Diabetes Care, 27: 1047-1053 (2004). The malfunction of glucose regulation arises from 1) insufficient secretion of insulin due to autoimmune-mediated destruction of pancreatic f3-cells (type 1 diabetes) or 2) disorders of both insulin resistance and secretion (type 2 diabetes) (Pickup, et al., Diabetes Metab. Res. Rev., 24: 604-610 (2008); Stumvoll, et al. Lancet, 365:1333-1346 (2005); and Kahn, Diabetes 43:1066-1084 (1994).

Frequent subcutaneous insulin injections and regular monitoring of blood glucose levels are essential for treatment of type 1 diabetic patients and some type 2 diabetic patients (Owens, et al., Lancet, 358:739-746 (2001)). However, such self-administration is painful and requires an indispensable commitment of patients. More importantly, this treatment, known as open-loop insulin delivery, does not maintain normoglycemia due to highly dynamic blood glucose concentrations (Jeandidier, et al., Adv. Drug Deliv. Rev., 35:179-198 (1999); Owens, et al., Nat. Rev. Drug Discov., 1:529-540 (2002)). Lack of tight control over glucose concentrations closer to the normal level accounts for many chronic complications such as limb amputation, blindness and kidney failure and can result in fatal hypoglycemia (N Engl J Med., 329:977-986 (1993)). Therefore, a pancreas-like, synthetic closed-loop device able to continuously and intelligently release insulin in response to blood glucose levels is highly desirable (Kumareswaran, et al. Expert Rev. Med. Devices, 6:401-410 (2009); Ravaine, et al., J. Control Release, 132:2-11 (2008)).

A straightforward strategy to achieve continuous release in response to glucose levels is to integrate a glucose monitoring moiety and a sensor-triggered insulin releasing moiety into one system. To date, a number of glucose-responsive formulations and devices have been explored, mainly derived from three categories: 1) glucose oxidase (GOx) based enzymatic reaction-induced response systems; 2) binding lectin protein Concanavalin A (Con A) based response systems, and 3) phenylboronic acid (PBA) based synthetic glucose-binding systems (Ravaine, et al., J. Control Release 132:2-11 (2008)).

A GOx based system is described in U.S. Pat. No. 4,364,385 to Lossef, et al., including a compartment isolated by a semipermeable, ionically charged membrane, containing glucose oxidase and catalase. U.S. Pat. No. 6,410,053 to Taylor discloses insulin immobilized in a dextran/concavalin A matrix, which can reversibly bind glucose and release insulin in response to changing glucose concentrations. PBA is boronic acid containing a phenyl substituent and two hydroxyl groups attached to boron. PBA and its derivatives form complexes with polyol molecules such as glucose and fructose, and can form stable hydrogels with a polyol such as poly(vinyl alcohol) (Hisamitsu, et al., Pharm. Res., 14:289-293 (1997)). The ability of PBA to bind polyols has been exploited in different ways to provide a glucose binding insulin delivery system. Hydrogels obtained from N-isopropylacrylamide (NIPAM) and PBA swelled and shrank according to the glucose concentration at pH 9. This system was modified to operate at physiological pH conditions by modifying the chemical structure of the receptor with an electron-withdrawing group on the phenyl ring. Matsumoto, et al., Biomolecules, 4(5):1410-6 (2003)). Other researchers have directly coupled a PBA moiety to insulin, to provide glucose binding insulin. For example, U.S. Publication No. 20030186846 by Hoeg-Johnson, et al., discloses an insulin delivery system made of insulin derivatives with a built in glucose sensor, such as an aryl boronate moiety.

These glucose insulin delivery systems have several limitations. The protein-involved platforms are not active for long periods due to denaturation under physiological conditions. The GOx based reaction needs additional dissolved oxygen. ConA exhibits significant cytotoxicity. These limit their implantable applications (Ravaine, et al., J. Control Release 132:2-11 (2008)). For PBA systems, the challenge remains to design devices that function in response to glucose at or near physiological pH.

It is therefore an object of this invention to provide a non-toxic, interactive or “smart” insulin delivery system that is responsive to changing glucose concentrations at or near physiological pH.

It is a further objection of the present invention to provide a method of controlling blood glucose levels in a patient in need thereof, by administering a smart insulin delivery system which responds to changing insulin concentrations at physiological pH.

### SUMMARY OF THE INVENTION

An injectable polymeric nanoparticle-crosslinked network formulation for controlled release of a therapeutic, prophylactic or diagnostic agent has been developed. The formulation includes particles formed of an acid-degradable polymer matrix, a component response to a physiological component such as glucose, and a therapeutic, prophylactic or diagnostic agent, wherein a first plurality of particles have a first nonzero charge on the surface and a second plurality of particles have a second opposite nonzero charge on the surface, which interact to form an injectable polymeric nanoparticle-crosslinked network, and the responsive acid component generates acid in the presence of a physiological component, which degrades the polymer to release the therapeutic, prophylactic or diagnostic agent.

An artificial “closed-loop” system able to mimic pancreas activity and release insulin in response to glucose level changes can improve patient compliance and health. The glucose-mediated release strategy for the self-regulated delivery of insulin uses an injectable and acid-degradable polymeric network formed by electrostatic interaction between oppositely charged polymeric nanoparticles such as dextran nanoparticles loaded with insulin and glucose-specific enzymes. The injectable polymeric nanoparticle-crosslinked network (designated nano-network) capable of glucose-mediated insulin delivery preferably uses chemically modified dextran (designated m-dextran) as an acid-degradable and biocompatible matrix material for glucose-regulated insulin delivery. Other matrix materials can be used, where degradation of the formulation is triggered by stimuli such as enzyme activity, redox conditions or photo irradiation. The composition contains a first plurality of particles and a second plurality of particles, both having insulin, glucose oxidizing enzyme (“GOx”), encapsulated in an acid-degradable polymeric matrix. The acid-degradable polymeric matrix is preferably a modified dextran polymer. The first plurality of particles further has a surface coating such as chitosan imparting a positive zeta potential. The second plurality of particles has a surface coating such as alginate imparting a negative zeta potential. In combination, the first plurality of particles and second plurality of particles form a nano-network gel, the gel being injectable under shear conditions and having rigidity under non-shear conditions. The nano-composite-based porous architecture dissociates to release insulin when present in a hyperglycemic state through the catalytic conversion of glucose into gluconic acid. In vitro insulin release can be modulated in a pulsatile profile in response to glucose concentrations.

In vivo studies show the formulations provide improved glucose control in Type 1 diabetic mice subcutaneously administered with the degradable nano-network. A single injection of the developed nano-network facilitated stabilization of the blood glucose levels in the normoglycemic state (<200 mg/dL) for up to at least 10 days.

Disclosed are injectable polymeric nanoparticle-crosslinked network formulations for controlled release of a therapeutic, prophylactic or diagnostic agent. The formulations can be include particles, where the particles include a responsive polymeric matrix, such as an acid-degradable polymer matrix, responsive signaling means, such as a responsive component or composition (examples of which include responsive signaling components, responsive acid components, and responsive acid signaling components), and a therapeutic, prophylactic or diagnostic agent. A first plurality of the particles have a first nonzero charge on the surface and a second plurality of the particles have a second opposite nonzero charge on the surface. The oppositely charged particles interact to form an injectable polymeric nanoparticle-crosslinked network. The responsive component generates acid in the presence of a physiological component and the acid degrades the polymer to release the therapeutic, prophylactic or diagnostic agent.

In some embodiments, the agent is insulin or insulin analog or an agent that increases insulin concentration, insulin levels, endogenous insulin, of combinations thereof. In some embodiments, the responsive signaling means is a responsive signaling component that includes glucose oxidase and catalase. This is an example of a glucose-responsive signaling component. In some embodiments, the ratio (w/w) of glucose oxidase and catalase to acid-degradable polymer matrix is from 1:100 to 1:15. In some embodiments, the glucose oxidase and catalase are present in a ratio (w/w) of 4:1.

In some embodiments, the first plurality of particles having a positive charge on their surface and the second plurality of particles having a negative charge on their surface interact to form a gel. In some embodiments, the zeta potential of the particles in the first plurality of particles and the zeta potential of the particles in the second plurality of particles have a magnitude from 5 to 15 mV. In some embodiments, the particles in the first plurality of particles can further include a surface modifier. In some embodiments, the surface modifier is chitosan or alginate. In some embodiments, the particles have a hydrodynamic radius of less than 350 nm.

In some embodiments, the responsive polymeric matrix is an acid-degradable polymer matrix and the acid-degradable polymer matrix includes a cross-linkable polymer and an acid-degradable cross linker. In some embodiments, the acid-degradable polymer matrix includes a polymer having a plurality of hydrolysable moieties. In some embodiments, the formulation dissociates under hyperglycemic conditions and does not substantially dissociate in normal glucose levels. In some embodiments, the formulation dissociates after 8 hours in a glucose concentration of 400 mg/dL. In some embodiments, the formulation does not substantially dissociate after 15 hours in normal glucose levels. In some embodiments, the release of the insulin or insulin analog or agent that increases insulin concentration is pulsatile when glucose concentrations are cyclically varied between normal and hyperglycemic conditions.

Also disclosed are methods of treating a patient in need thereof comprising administering to an individual in need thereof an effective amount of injectable polymeric nanoparticle-crosslinked network formulations as disclosed herein. In some embodiments, the agent is insulin or insulin analog or agent that increases insulin concentration and the individual has type 1 or type 2 diabetes. In some embodiments, the formulation is administered to maintain normoglycemia, normal glycalated albumin levels, or higher body condition score. In some embodiments, an amount of the formulation effective to maintain blood glucose concentrations at a between 70-130 mg/dL or 90-110 mg/dL is administered.

### BRIEF DESCRIPTION OF THE DRAWINGS

FIG. 1A depicts a chitosan coated m-dextran nanoparticle (NP) on the left and an alginate coated m-dextran nanoparticle (NP) on the right, each containing encapsulated insulin, glucose oxidase (GOx), and catalase (CAT). FIG. 1B depicts the structure of the acetal modified dextran polymer m-dextran. FIG. 1C depicts the formation of the nano-network (NN) gel upon mixing the chitosan coated particles and the alginate coated particles. GOx converts glucose to gluconic acid, thereby lowering the pH. The NN and the NPs degrade thereby releasing insulin. FIG. 1D depicts subcutaneous injection of the NN gel into a diabetic mouse in hyperglycemia. The glucose-mediated NN degradation promotes normoglycemia.

FIG. 2 is a schematic of synthesis and acid-degradation of m-dextran.

FIG. 3A is a schematic of enzymatic reactions through glucose oxidase (GOx) and catalase (CAT). FIG. 3B is a graph of the pH over time (minutes), showing the decrease in 0.5 mL 400 mg/dL glucose saline solution at 37° C. in the presence of GOx and CAT with different weight ratios.

FIGS. 4A and 4B are graphs of cell viability (percent) as a function of concentration (micrograms/ml) of empty m-dextran nanoparticles coated with chitosan or alginate (FIG. 4A) and their degradation products (FIG. 4B) after 24 hour culture with HeLa cells.

FIG. 5 is a graph of the viscosity and shear-thinning behaviors of the nano-network (NN(E+I)) upon the incubation with 400 mg/dL glucose at 37° C. over time.

FIGS. 6A-6C are graphs showing glucose-responsive degradation of nano-network and insulin release. FIG. 6A is a graph of relevant pH changes in different incubation solutions with nano-networks; FIG. 6B is a graph of the in vitro accumulated insulin release of nano-network in different glucose concentrations at 37° C.; and FIG. 6C is a graph of the self-regulated profile of nano-network presents the rate of insulin release as a function of glucose concentration. Data points represent mean±SD (n=2) in a), d) and e).

FIGS. 7A-7D are graphs of in vivo studies of nano-network for Type 1 diabetes treatment. Blood glucose levels (7A) and plasma human insulin concentration (7C) in STZ-induced C57B6 diabetic mice after subcutaneous injection with 1×PBS, nano-network encapsulated insulin and enzymes (NN(E+I)), nano-network encapsulated insulin only (NN(I)), nano-network encapsulated with enzymes only (NN(E)) or pure insulin solution. Changes of the mice number in different groups within the normoglycemic range (<200 mg/dL) over the administration time are shown in FIG. 7B. The glycated albumin percentages of mice treated with PBS, NN(E+I), NN(I), NN(E) and insulin solution are shown in FIG. 7D. Student's t-test: *p<0.05, ***p<0.001. Data points represent mean±SD (n=8).

FIG. 8 is a graph of 12-hour monitoring of blood glucose levels in STZ-induced C57B6 diabetic mice after subcutaneous injection with 1×PBS, nano-network encapsulated with insulin and enzymes (NN(E+I)), nano-network encapsulated with insulin only (NN(I)), nano-network encapsulated with enzymes only (NN(E)) or pure insulin solution.

FIG. 9 is a graph showing the in vivo glucose-responsiveness of the nano-network. 6 days after injection of NN(E+I), an i.v. glucose tolerance test was performed (1.5 g/kg body weight) in the group treated with NN(E+I) and compared with healthy mice. Data are expressed as mean±SD (n=5) FIG. 10 is a graph showing in vivo biocompatibility, with a graph of changes of lump sizes in the injection sites of STZ-induced C57B6 diabetic mice treated with NN(E+I) and NN(I) over time.

### DETAILED DESCRIPTION OF THE INVENTION

### I. Definitions

“Hydrophilic,” as used herein, refers to molecules which have a greater affinity for, and thus solubility in, water as compared to organic solvents. The hydrophilicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the water than in the organic solvent, then the compound is considered hydrophilic.

“Hydrophobic,” as used herein, refers to molecules which have a greater affinity for, and thus solubility in, organic solvents as compared to water. The hydrophobicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the organic solvent than in the water, then the compound is considered hydrophobic.

“Hydrogel,” as used herein, refers to a gelatinous colloid, or aggregate of polymeric molecules in a finely dispersed semi-solid state, where the polymeric molecules are in the external or dispersion phase and water (or an aqueous solution) is forms the internal or dispersed phase. Generally, hydrogels are at least 90% by weight of an aqueous solution.

“Peptide,” as used herein includes “polypeptide,” “oligopeptide,” and refers to a chain of at α-amino acid residues linked together by covalent bonds (e.g., peptide bonds). The length of the peptide is limited at the lower end only by the minimum number amino acids required to form a self-assembling peptide.

“Small Molecule”, as used herein, refers to a molecule, such as an organic or organometallic compound, with a molecular weight of less than 2,000 Daltons, less than 1,500 Daltons, less than 1,000 Daltons, less than 750 Daltons, or less than 500 Daltons. The small molecule can be a hydrophilic, hydrophobic, or amphiphilic compound.

The term “oligomeric”, as used herein, describes something made primarily from a plurality of monomeric units and is generally referred to as an “oligomer”. An oligomer can have a molecular weight between 10 Daltons and 15,000 Daltons, between 100 Daltons and 10,000 Daltons, or between 500 Daltons and 5,000 Daltons. An oligomer can have from 3 to 100 monomeric units, from 4 to 50 monomeric units, or from 5 to 25 monomeric units.

The term “enzyme”, as used herein, can generally refer to one or a combination of enzymes. It is to be understood that enzyme variants (produced, for example, by recombinant techniques) are included within the meaning of the term “enzyme”.

The term “polymeric”, as used herein, describes something made primarily from a “polymer”. The term “polymer” is generally recognized in the art. The term “polymeric” should be broadly construed to include, without limitation, homopolymers, copolymers, terpolymers, and the like as well as interpolymers, and blends and combinations of all of the above. As used herein, polymeric components typically have a molecular weight greater than 1 kDa, greater than 5 kDa, or greater than 10 kDa.

The terms “incorporated” and “encapsulated” refers to incorporating, formulating, or otherwise including an active agent into and/or onto a composition that allows for release, such as sustained release, of such agent in the desired application. The terms contemplate any manner by which a therapeutic agent or other material is incorporated into a polymer matrix, including, for example: attached to a monomer of such polymer (by covalent, ionic, or other binding interaction), physical admixture, enveloping the agent in a coating layer of polymer, incorporated into the polymer, distributed throughout the polymeric matrix, appended to the surface of the polymeric matrix (by covalent or other binding interactions), encapsulated inside the polymeric matrix, etc. The term “co-incorporation” or “co-encapsulation” refers to-the incorporation of a therapeutic agent or other material and at least one other therapeutic agent or other material in a subject composition.

“Nanoparticle”, as used herein, generally refers to a particle of any shape having a diameter from about 1 nm up to, but not including, about 1 micron, preferably from 3 nm to about 500 nm. Nanoparticles having a spherical shape are generally referred to as “nanospheres”. The size of nanoparticles can be experimentally determined using a variety of methods known in the art, including transmission electron microscopy (TEM), dynamic light scattering (DLS), gel filtration chromatography (GFC), and fluorescence correlation spectroscopy (FCS).

“Biocompatible” and “biologically compatible”, as used herein, generally refer to materials that are, along with any metabolites or degradation products thereof, generally non-toxic to the recipient, and do not cause any significant adverse effects to the recipient. Generally speaking, biocompatible materials are materials which do not elicit a significant inflammatory, immune or toxic response when administered to an individual.

“Biodegradable polymer” and “bioerodible polymer” are used herein interchangeably, and generally refers to a polymer that will degrade or erode by enzymatic action or hydrolysis under physiologic conditions to smaller units or chemical species that are capable of being metabolized, eliminated, or excreted by the subject. The degradation time is a function of polymer composition, morphology, such as porosity, particle dimensions, and environment. Suitable degradation times are from hours to weeks, more preferable from hours to days. For example, the polymer may degrade over a time period from one hour to fourteen days, from three hours to seven days, from twelve hours to seven days, or from eighteen hours to two days.

The term “zeta potential” is used herein to mean without limitation a potential gradient that arises across an interface. This term especially refers to the potential gradient that arises across the interface at the surface of a nanoparticle, also referred to as surface charge. Migration velocity of the particles depends on the amount of the surface charges and the applied field strength. Particles having a positive zeta potential migrate toward the negative electrode, and likewise particles having a negative zeta potential migrate toward the positive electrode. To determine the rate of migration, migrating particles are irradiated with a laser in the electric field. The movement of the particles is measured in a frequency shift in the reflected light compared to the incident light. The amount of frequency shift is dependent on the migration speed and is the so-called Doppler frequency shift (Doppler effect). From the Doppler frequency, the wavelength, the scattering angle and the rate of migration of a particle can be derived. The electrophoretic mobility is determined by the ratio of the moving speed and the electric field strength. The zeta potential is directly proportional to the electrophoretic mobility and is typically reported in mV. “Zeta potential”, as used herein, is defined wherein the zeta potential and particle size distribution were measured by dynamic light scattering (DLS) using a 90Plus Particle Size Analyzer by Brookhaven Instruments.

The terms “smart delivery system” or “interactive delivery system”, as used interchangeably herein, refer to a delivery system for one or more therapeutic, prophylactic, or diagnostic agents wherein the rate of delivery is responsive to one or more stimuli indicative of the need for delivery, i.e. as a non-limiting example a smart insulin delivery system delivers insulin at a rate that is dependent upon the glucose levels in proximity to the delivery system.

The total response, i.e. the total amount of insulin released or the total amount of degradation of the polymeric matrix will depend upon the total time the pH is acidic enough to promote degradation, i.e. the amount of time it takes to restore normoglycemia. The response is preferably pulsatile, and preferably little to no insulin is released (there is little to no degradation) at physiological pH 7.4. The acid degradable polymers described herein should have a significantly lower rate of degradation in solution at pH 7.4 than at pH 5. In a preferred embodiment, the polymers should have a degradation half-life at pH 5.0 of 5 minutes to 24 hours at 37° C., but a longer half-life at pH 7.4 of at least 12 hours to 250 days. In some embodiments, it may be useful for the polymers to have a half-life at pH 5.0, 37° C. of about 5-30 minutes, of about 2-5 hours, or of about 24 hours, while a half-life at pH 7.4, 37° C. of about 90 days, about 180 days, or about 250 days, in order to facilitate the rapid release of bioactive materials at acidic pH and slow release of bioactive materials at physiological pH. In some embodiments, the modified polyhydroxylated polymers are largely stable at pH higher than 7.4 but hydrolyze at a pH preferably about 5.

The term “pulsatile” or “pulsatile release”, as used herein, refers to the release of multiple doses from a single administration to a subject. The individual doses can be administered at a variety of intervals, depending on the formulation of the delivery system and the application. A smart pulsatile delivery system is capable of administering multiple doses of a therapeutic, prophylactic, or diagnostic agent in response to one or more stimuli, preferably wherein the dosage delivered is responsive to the deviation of the stimuli from a target value. As a non-limiting example, a smart pulsatile insulin delivery system preferably delivers little to no insulin during periods of normoglycemia but delivers a dosage of insulin in response to hypoglycemic conditions that is responsive to the deviation from normoglycemia, preferably in an amount sufficient to restore normoglycemic glucose levels.

The amount released depends upon the pH and the time to locally restore physiological pH for the example pH responsive insulin delivery system, more generally the amount released should depend upon the deviation of the external stimulus from the normal value and the time needed to return to the normal value.

The term “ligand and acceptor pair”, as used herein, can refer to any combination of biocompatible ligand and acceptor wherein the ligand associates either exclusively or primarily with the acceptor. The association can include, but is not limited to, one or more of non-covalent (i.e., for example, ionic, Van der Waals forces, electrostatic, etc.) or covalent interactions. The ligand and/or the acceptor can be naturally occurring or can be synthetic. The ligand can be a nucleic acid, polypeptide, peptide, glycoprotein, glycopeptide, proteoglycan, carbohydrate, lipid, small molecule, etc. The receptor can be a naturally occurring receptor such as a cell surface receptor or an analog or derivative thereof, or can be a synthetic receptor. The receptor can be a protein, a carbohydrate, a lipid, and/or a nucleic acid. The ligand and acceptor pair preferably bind with high affinity. A ligand and acceptor pair binding with “high affinity” bind with a dissociation constant less than 1 μM, less than 10 nM, less than 1 nM, or less than 100 pM.

### II. Smart Nano-Network for Controlled, Responsive Insulin Release

An artificial pancreas-like synthetic closed-loop device able to continuously and intelligently release insulin in response to blood glucose levels has been developed. This injectable gel-like nano-network uses a material such as the acid-degradable and biocompatible dextran as a matrix material, for long-term delivery of insulin. By incorporating glucose oxidase, the nano-network can be effectively dissociated under hyperglycemia conditions, which is suitable for development of self-regulation based insulin delivery system in a glucose-responsive fashion.

The bulk polymeric matrix used in this approach is highly biocompatible and biodegradable. Representative polymers include dextran, poly(lactic-co-glycolic acid) (PLGA), hyaluronic acid (HA), chitosan, alginate and poly(beta-amino ester) (PBAE);

The nano-network with payload can be directly administrated through subcutaneous injection;

The small size of nanoparticles as well as the porous structure of nano-network provides large surface area for thorough contact of reagents as well as easy diffusion;

The nano-network structure can adjust drug release with near zero-order kinetics;

The encapsulation efficiency of payload (insulin) through nano-network is high (i.e., 40-60%); and

The degradability of matrix materials can be tailored to adjust release profiles.

From a functional perspective, as demonstrated in the examples:

The insulin in vitro release profile of the nano-network exhibits a prominent pulsatile pattern: with a high release rate at a hyperglycemic level (400 mg/dL) and low release rate at a normal level (100 mg/dL), which is highly desirable for self-regulation based closed loop delivery systems;

The nano-network displays sustained in vivo insulin release as well as long-term pharmacological duration (up to 7-10 days) of insulin activity release. The active duration can be further improved through adjusting injection doses as well as degradation properties or matrix materials.

This nano-network based platform for long-term and glucose-responsive delivery of insulin, when optimized, has advantages over the current approaches for closed-loop insulin delivery. It is a competitive delivery tool for both smart and safe intentions. Moreover, besides transporting insulin, this platform can also be extended to delivery or co-delivery of other therapeutic agents, including insulin analogs, other proteins/peptides, and small molecular anti-inflammation drugs. Considering the considerable market size of devices and therapies for diabetes management, a strategy identified with sustained and long-term release, intelligent response, ease of administration, good biocompatibility and facile preparation will definitely generate extensive interest

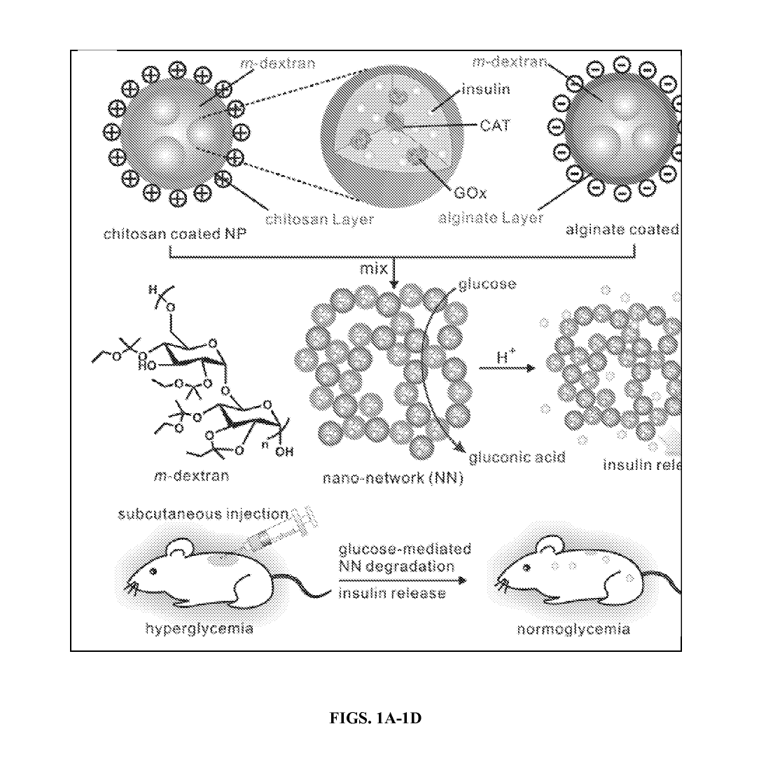

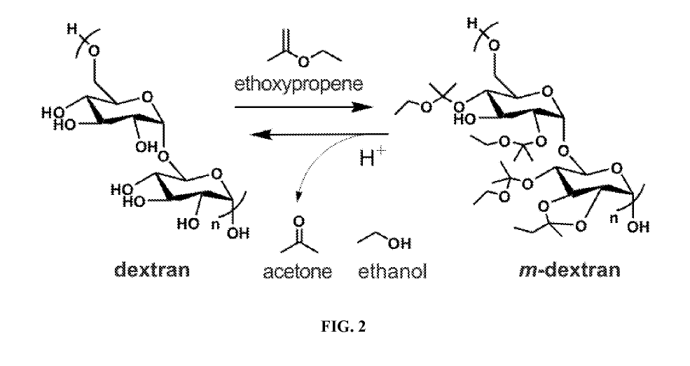

A schematic of the nano-network is shown in FIGS. 1A-1C. Oppositely charged degradable nanoparticles interact and self-assemble through electrostatic forces to create a cohesive gel-like network. The produced gel network forms a stable three-dimensional, porous structure, which yields an increased surface area to volume ratio for maximum interaction between glucose and GOx (FIG. 2B). Materials of this design should release payloads with near zero-order kinetics25. Additionally, the nano-network produces shear-thinning behavior due to the disruption of particle-particle interactions as the applied shear force is increased. Once the external force is withdrawn, the strong cohesive property is recovered, which allows for convenient molding and injection (FIGS. 1A-1B).

The examples show a gel formed from two types of nanoparticles, one type formed from modified dextran nanoparticles where the hydroxy groups are converted to tertiary ethers with 2 ethoxypropene, and the other type formed of alginate. The particle surfaces are coated with chitosan or alginate to produce positive or negative charges on the surface respectively. The charged particles then form the gel. The particles are loaded with insulin and GOx/CAT (glucose oxidase/catylase). The GOx/CAT system converts glucose into a local pH change. The modified polymers are pH-responsive.

A. Responsive Polymeric Matrix

The bulk polymeric matrix should be highly biocompatible and biodegradable. The bulk polymeric matrix should have a rate of biodegradation that is responsive to one or more external stimuli. External stimuli can include pH, temperature, concentration of one or more chemical or enzymatic agents, radiation, etc. In a preferred set of embodiments the rate of biodegradation of the bulk polymeric matrix is responsive to the local pH.

Representative polymers include homopolymers and copolymers of polysaccharides such as alginate, chitosan, dextran, mannan, pullulan, hyaluronic acid (HA), and xanthan gum; biodegradable polyesters such as polylactic acid, polyglycolic acid, poly(3-hydroxybutyrate), and polycaprolactone; acrylate and methacrylate polymers such as 2-(hydroxyethyl) methacrylate, and copolymers thereof. The rate of biodegradation can in some embodiments be adjusted by altering the ratio of repeat units in a copolymer. For example, when the polymeric matrix is poly(lactic-co-glycolic acid) (PLGA), the rate of biodegradation can be controllably varied from a few days to several months by varying the ratio of lactic acid and glycolic acid in the polymer. The polymers forming the polymeric matrix can in some embodiments be modified to provide or to enhance responsiveness to the one or more external stimuli. These are preferably hydrophilic polymers. For example, in some embodiments a biodegradable polymer such as a polysaccharide is modified to be pH responsive.

In preferred embodiments the rate of biodegradation of the bulk polymeric matrix is responsive to local pH. Examples of pH-sensitive polymers useful in drug delivery include polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinylacetate phthalate copolymer, styrene and maleic acid copolymers, polyacrylic acid derivatives such as acrylic acid and acrylic ester copolymers, polymethacrylic acid and esters thereof, poly acrylic methacrylic acid copolymers, shellac, and vinyl acetate and crotonic acid copolymers.

In preferred embodiments the polymer forming the bulk polymeric matrix is modified to provide or to increase responsiveness to the local pH. For example, in some embodiments the polymer is a modified polyhydroxylated polymer that is a polyhydroxylated polymer having reversibly modified hydroxyl groups, wherein the hydroxyl groups are modified to feature an acid degradable functional group. Exemplary acid degradable functional groups can be acetals, aromatic acetals, ketals, vinyl ethers, aldehydes, or ketones. The hydroxyl groups in the polyhydroxylated polymers are modified, thereby rendering the modified polyhydroxylated polymer acid degradable, pH sensitive and typically insoluble in water. The polyhydroxylated polymers can be preformed natural polymers or hydroxyl-containing polymers including, but not limited to, multiply-hydroxylated polymers, polysaccharides, carbohydrates, polyols, polyvinyl alcohol, poly amino acids such as polyserine, and other polymers such as 2-(hydroxyethyl)methacrylate. Exemplary polysaccharides that can be used to form modified polyhydroxylated polymers include, but are not limited to, dextran, mannan, pullulan, maltodextrin, starches, cellulose and cellulose derivatives, gums (e.g., xanthan, locust bean, etc.), and pectin. In one embodiment, the polysaccharides are dextran or mannan.

The reversible modification of the hydroxyl groups in modified polyhydroxylated polymers can be carried out to provide modified hydroxyl groups, wherein at least 20%, 25%, 30%, 40%, 50%, 60%, 70%, 75%, 80%, 85%, 90%, 95%, 98%, 99% or 100% of the hydroxyl groups in the polymer are modified.

The choice of the polyhydroxylated polymer and the degree of modification can be based upon such factors as ease of synthesis, solubility, commercially available reagents, the type of acid-degradable polymer desired, the loading efficiency, dispersion of drug delivery systems comprised of the polymers, toxicity and the hydrolysis rates of the acetal linkage. It is preferred that the degradation products are biocompatible and biodegradable. For example, the degradation products should be non-immunogenic and non-toxic, for example, with the size and/or toxicity levels preferred by one having skill in the art for approved in vivo use.

The modified polyhydroxylated polymers can be modified polysaccharides with pendant acetals, thus providing acetal-derivatized polysaccharides. In some embodiments, the modified polyhydroxylated polymers are acetal-derivatized dextran, acetal-derivatized mannan or acetal-derivatized polyvinyl alcohols, preferably acetal-derivatized dextran.

The modified polymers having a modified functional (e.g., acetal or ketal) linkage at the modified hydroxyl groups should degrade by acid catalyzed hydrolysis into lower molecular weight compounds that can be completely excretable. The rate of hydrolysis of these polymers can be changed by varying the functional group (e.g., acetal or ketal) linkage from slow degrading to fast degrading, the degree of modification, or the hydrophobicity of the modification, thus providing a wide range of release kinetics for drug delivery. Thus, it is contemplated that a variety of acid degradable linkages with different acid-sensitivities can be incorporated onto the polymer backbones, allowing for greater control of the rate of polymer hydrolysis.

In a preferred embodiment, the present acid degradable polymers described herein should have a significantly lower rate of degradation in solution at pH 7.4 than at pH 5. In a preferred embodiment the polymers should preferably have a degradation half-life at pH 5.0 of 5 minutes to 24 hours at 37° C., but a longer half-life at pH 7.4 of at least 12 hours to 250 days. In some embodiments, it may be useful for the polymers to have a half-life at pH 5.0, 37° C. of about 5-30 minutes, of about 2-5 hours, or of about 24 hours, while a half-life at pH 7.4, 37° C. of about 90 days, about 180 days, or about 250 days, in order to facilitate the rapid release of bioactive materials at acidic pH and slow release of bioactive materials at physiological pH. In some embodiments, the modified polyhydroxylated polymers are largely stable at pH higher than 7.4 but hydrolyze at a pH preferably about 5. In one embodiment, the modified polymers are soluble in common organic solvents to facilitate processing into a variety of materials. In another embodiment, these modified polymers are not water soluble.

Other matrix materials can be used, where degradation of the formulation is triggered by other stimuli, such as enzyme activity, redox conditions or photo irradiation.

B. Responsive Signaling Means

In some embodiments, the bulk polymeric matrix will have a rate of degradation that is responsive to the external stimulus of interest. For example, a composition designed to deliver a bioactive agent upon detection of a particular enzyme can contain a polymer with a rate of biodegradation that increases upon the presence of that particular enzyme. In other embodiments, the compositions can contain a responsive signaling means that converts the desired stimulus into a local change that impacts the biodegradation of the polymeric matrix. For example, compositions containing a pH responsive polymeric matrix can contain a signaling means capable of detecting the presence of a particular analyte and locally altering the pH in response. In preferred embodiments the composition contains a glucose-responsive signaling means capable of locally altering one or more parameters in response to glucose concentration. For example, compositions are provided containing a pH responsive polymeric matrix and a signaling component capable of locally altering the pH in response to blood glucose levels.

The enzymes may be derived from any suitable source, such as, a bacteria, a fungus, or a yeast. The types of enzymes which may appropriately be incorporated as a responsive signaling means include oxidoreductases, transferases, hydrolases, lyases, isomerases, and ligases. Preferred enzymes include oxidoreductases such as glucose oxidases, peroxidases, and laccases as well as catalases. One skilled in the art will recognize the total amount of responsive signaling means will depend upon the amount of responsive polymeric matrix and the activity level of the signaling means. In some embodiments the responsive polymeric matrix contains an acid degradable polymer and the GOx/CAT signaling means described above, preferably wherein the ratio of total enzymes to polymer (w/w) is from 1:1000 to 1:1, preferably from 1:500 to 1:2, more preferably from 1:100 to 1:15. The glucose oxidase can in principal be any biocompatible enzyme displaying glucose oxidase activity that as so long as it is capable of oxidizing glucose to generate gluconic acid, preferably also producing peroxide when used in combination with a catalase. Commercially available glucose oxidases include GLUZYME™ 2.500 BG, GLUZYME™ 10000 BG and GLUZYME™ MONO 10000 BG, available from Novozymes A S, Denmark, FERMIZYME™ GO 10.000 and FERMIZYME™ GO 1500 available from DSM, HYDERASE™ 15 and HYDERASE™ HC available from Amano, or OXYGO® available from Genencor Int., Commercially available catalases include TERMINOX™ and TERMINOX Ultra™ (Novozymes A/S, Bagsvaerd, Denmark) and Catalase T100T™ (Genencor International Inc.).

Glucose oxidase (GOx) is an enzyme which catalyzes the conversion of glucose to gluconic acid in the presence of oxygen shown in the following reaction

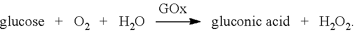

Previous glucose-responsive systems with GOx typically entrap or immobilize the enzyme within biomaterials, causing a local decrease in pH with increasing glucose concentrations. See for example Gordijo et al., Adv. Funct. Mater. 2011, 21(1):73-82; Fischel-Gordon et al., Proc. Nat. Acad. Sci. USA 1988, 85(7):2403-2406; and Traitel et al., Biomaterials 2000, 21(16):1679-1687. Based on this strategy a number of bulk hydrogel systems have been developed such as those described in Bratlie et al., Adv. Healthcare Mater. 2012, 1:267-284 and Ravaine et al., J. Control. Release 2008, 132(1):2-11. However, bulk hydrogel systems such as these exhibit slow response to changes in glucose concentration due to mass transport limitations. On the other hand, GOx containing membranes suffer from poor mechanical strength resulting in premature insulin leakage.

The colloidal hydrogel materials are capable of providing rapid responses to changes in glucose concentration. The compositions may contain both GOx and catalase (CAT). The catalase enzyme works in conjunction with the GOx enzyme (as depicted in FIG. 3A) by providing O₂ consumed in the GOx mediated oxidation of glucose to gluconic acid. The H₂O₂ produced is converted back into O₂ by CAT, providing a source of oxygen and an increased driving force for the conversion of glucose.

The total amount of enzymes will depend upon multiple factors, especially the total amount of polymeric matrix and the activity level of the enzyme.

Example 1 has 3.5 mg of total enzymes (GOx and Cat) and 240 mg of the modified dextran. One skilled in the art will recognize the total amount of responsive signaling means will depend upon the amount of responsive polymeric matrix and the activity level of the signaling means. In some embodiments the responsive polymeric matrix contains an acid degradable polymer and the GOx/CAT signaling means described above, preferably wherein the ratio of total enzymes to polymer (w/w) is from 1:1000 to 1:1, preferably from 1:500 to 1:2, more preferably from 1:100 to 1:15. The glucose oxidase can in principal be any biocompatible enzyme displaying glucose oxidase activity that is capable of oxidizing glucose to generate gluconic acid, preferably also producing peroxide when used in combination with a catalase.

In some embodiments the weight ratio of GOx to CAT is optimized to enhance the capability of the enzyme mixture to respond to changing glucose levels. The weight ratio of GOx to CAT can be from 1:100 to 100:1, although preferably the ratio is from 1:1 to 8:1. In some embodiments, the response of the GOx/CAT system is optimized at a weight ratio of GOx to CAT of about 4:1.

The responsive signaling means can be incorporated into the polymeric matrix, can be coated onto the surface of the particles, can be encapsulated within a different biodegradable particle, or be administered separately from the composition. In preferred embodiments the responsive signaling means is incorporated within the composition, preferably within the responsive polymeric matrix.

The responsive signaling means can contain one or more enzymes such as the GOx/CAT signaling means described above. Enzymes suitable for this purpose can be any enzymes. Suitable enzymes include hydrolases, cutinases, oxidases, transferases, reductases, hemicellulases, esterases, isomerases, pectinases, lactases, peroxidases, laccases, pectinases, catalases, nitrilases and mixtures thereof. Hydrolases hydrolyze substrates and include, but are not limited to, proteases (bacterial, fungal, acid, neutral or alkaline), amylases (alpha or beta), lipases, phospholipases, esterases, mannanases, cellulases, and mixtures thereof. Particular enzymes of interest are oxidases, catalases, peroxidases, and dehydrogenases.

In some embodiments responsive hydrogel compositions are provided containing one or more oxidases in combination with CAT. Examples of enzymes that can be used, optionally in combination with CAT, include glucose oxidase, α-hydroxy oxidase, lactate oxidase, urease, creatine amidohydrolase, creatine amidinohydrolase, sarcosine oxidase, glutamate dehydrogenase, pyruvate kinase, alcohol oxidase, lactate dehydrogenase, and fructose dehydrogenase. For example, a hydrogen composition containing a pH responsive polymeric matrix and the enzyme combination of alcohol oxidase and catalase can be used to deliver one or more active agents in response to blood alcohol levels.

C. Surface Modifiers

In some embodiments, the particles are coated with a surface modifier. A surface modifier can be applied to create particles having a charge or to create particles having different charges, i.e. to create two or more pluralities of particles wherein one plurality of particles may have a greater magnitude of charge (more negative or more positive) or may have an opposite charge as compared to a different plurality of particles. Nanoparticles of opposite charge can be used to create a gel, as shown in FIGS. 1A and 1B. Surface modifiers can be small molecule, oligomeric, or polymeric in nature. Surface modifiers can modify one or more properties related to charge, charge density, zeta potential, mechanical strength, rigidity, color, surface roughness, magnetic moment, or the presence and density of moieties on the surface. The moieties can include moieties that create specific or non-specific attractive (binding) interactions between particles. Exemplary moieties can include ligand and acceptor pairs such as antigen/antibody pairs, hydrogen bond donors and hydrogen bond acceptors, and cross-linking moieties. For example, a first plurality of particles can have a surface modifier presenting a plurality of acceptor moieties where a second plurality of particles can have a surface modifier presenting a plurality of targeting ligands that specifically bind the acceptor, thereby creating a strong attractive interaction between the different particles. Likewise, particles having a surface presenting a plurality of hydrogen bond donors will have a strong attractive interaction to particles presenting a plurality of hydrogen bond acceptors.

In some embodiments the surface modifiers include one or more ligand/acceptor pairs, preferably binding with high affinity. High affinity ligand/acceptor pairs are known in the literature. Exemplary high affinity ligand/acceptor pairs include FK506/FKBP12, methotrexate/dihydrofolate reductase, PPI-2458/methionine aminopeptidase, biotin/streptavidin tetramer, hirudin/thrombin, ZFV^{P}(O)F/carboxypeptidase, and chloroalkanes/haloalkane dehalogenases. Methods of determining high affinity binding pairs are known and can be used to identify high affinity ligand/acceptor pairs.

In some embodiments, the surface modifiers can include one or more hydrogen bond donors and/or one or more hydrogen bond acceptors. Exemplary hydrogen bond donors include moieties having available hydroxy or amino groups, including alcohols, phenols, carboxylic acids, primary and secondary amines, phosphonic acids, phosphoric acid esters, sulfonic acids, and sulfuric acids. Monosaccharides contain free —OH groups, therefore monosaccharides, disaccharides, oligosaccharides, and polysaccharides are exemplary hydrogen bond donors. Exemplary surface modifiers containing sugars include polymers such as alginate, chitosan, polyvinylalcohol, cellulose and cellulose derivatives such as methylcellulose, hydroxypropyl cellulose, hydroxyethyl cellulose, hydroxyethylmethyl cellulose, carboxymethyl cellulose, cellulose acetate, cellulose acetate phthalate, croscarmellose, hypromellose, and hydroxypropyl methyl cellulose), carrageenan, cyclodextrins, dextrin, polydextrose, other starches like corn starch, amylase, amylopectin, and sodium starch glycolate, sugars or small molecules like malic acid, trehalose, propylene glycol, glycerol, glycerol monostearate, sugars like sorbitol, ribose, arabinose, xylose, lyxose, allose, altrose, mannose, mannitol, glucose, dextrose, idose, galactose, talose, glucose, fructose, dextrose, dextrates, lactose, sucrose, and maltose, stearic acid, Vitamin E, and derivatives thereof. Exemplary hydrogen bond acceptors contain electronegative groups such as oxygen, nitrogen, sulfur, etc. with free valence electron pairs, including moieties containing for example —CO— and ═N—. Exemplary hydrogen bond acceptors include nitrogen containing groups such as amines, amides, imines, imides, nitriles and ureas as well as aromatic nitrogen-based functional groups such as pyridines, imidazoles, etc. as well as carboxylate groups (carboxylic acid, carboxylic ester), phosphonates, sulfoxides, sulfones, and carbamates.

While in some embodiments the nanoparticles are made of polymers having opposite charge at physiological pH, in other embodiments, the nanoparticles are made of the same polymer and one or more groups of particles are coated with a biocompatible material having a positive charge or a negative charge. For example, to obtain oppositely-charged nanoparticles, polysaccharides such as chitosan and alginate, two polysaccharides that are used in humans, can be employed as surface modifiers to coat the nanoparticles to introduce both a positive and negative, respectively, surface, as shown in FIG. 1B.

In some embodiments, different groups of particles can have opposite charges (opposite in sign) or can have charges that are of the same sign but having a different magnitude or amount of charge on the surface. In some embodiments particles having an opposite charge, either by nature of the polymeric matrix having charge or via the charges from surface modifiers, can be combined to form a gel. The charged surface modifiers can include small molecules, oligomers, or polymers having a positive or a negative charge, preferably at or near a physiological pH. In some embodiments the charged surface modifier can be a surfactant on the surface of the particle. Exemplary positively charged surfactants (cationic surfactants) can include benzalkonium chloride (alkylbenzyldimethylammonium chloride); cetylpyridinium chloride; and cetyltrimethylammonium chloride (hexadecyltrimethylammoniurn chloride). Exemplary negatively charged surfactants (anionic surfactants) can include Dilauroylphosphoglycerol (1,2-Dilauroyl-sn-Glycero-3-[Phospho-rac-(1-glycerol)]; phosphatidic acid; saturated fatty acids, such as lauric acid, myristic acid, palmitic acid and stearic acid; unsaturated fatty acids, such as palmitoleic acid, oleic acid, linoleic acid and linolenic acid; deoxycholic acid; cholic acid; caprylic acid; glycocholic acid; glycodeoxycholic acid; lauroylsarcosine; and n-dodecyl sulfate.

In some embodiments particles are coated with surface modifiers containing one or more crosslinkers, for example, a thermally activated or UV activated crosslinker. Such crosslinkers include thermal crosslinkers which are activated at physiological temperatures or upon the application of heat. Such thermal crosslinkers can include multifunctional isocyanates, aziridines, multifunctional (meth)acrylates, and epoxy compounds. Exemplary crosslinkers include difunctional acrylates such as 1,6-hexanediol diacrylate or multifunctional acrylates such as are known to those of skill in the art. UV activated crosslinkers can also be used to crosslink the particles. Such UV crosslinkers can include benzophenones and 4-acryloxybenzophenones.

In some embodiments the particles are made from or are coated with surface modifiers that are charged polymers, i.e. cationic polymers or anionic polymers. Exemplary cationic polymers include linear and branched homopolymers and copolymers of polyallylamine (PAH); polyethyleneimine (PEI); poly(L-lysine) (PLL); a poly(L-arginine) (PLA); polyvinylamine; poly(vinylbenzyl-tri-Ci-C4-alkylammonium salt); poly(vinylpyridin), a poly(vinylpyridinium salt); a poly(N,N-diallyl-N,N-di-Ci-C4-alkyl-ammoniumhalide); and/or polyaminoamide. Exemplary cationic polymers can include the copolymer of hydroxyethyl cellulose and diallyldimethylammonium chloride, the copolymer of acrylamide and diallyldimethylammonium chloride, the copolymer of vinyl pyrrolidone and dimethylamino ethylmethacrylate methosulfate, the copolymer of acrylamide and betamethacrylyloxyethyl trimethyl ammonium chloride, the copolymer of polyvinyl pyrrolidone and imidazolimine methochloride, the copolymer of diallyldimethyl ammonium chloride and acrylic acid, the copolymer of vinyl pyrrolidone and methacrylamidopropyl trimethyl ammonium chloride, the methosulfate of the copolymer of methacryloyloxyethyl trimethylammonium and methacryloyloxyethyl dimethylacetylammonium, quaternized hydroxyethyl cellulose; dimethylsiloxane 3-(3-((3-cocoamidopropyl)dimethylammonio)-2-hydroxyprpoxy)propyl group terminated acetate; the copolymer of aminoethylaminopropylsiloxane and dimethylsiloxan; the polyethylene glycol derivative of aminoethylaminopropylsiloxane/dimethylsiloxan-copolymer and cationic silicone polymers. Exemplary anionic polymers include linear and branched homopolymers or copolymers of polyacrylic acid (PAA), polymethacrylic acid (PMA), maleic acid, fumaric acid, poly(styrenesulfonic acid) (PSS), polyamido acid, poly(2-acrylamido-2-methylpropanesulfonic acid) (poly-(AMPS)), alkylene polyphosphate, alkylene polyphosphonate, carbohydrate polyphosphate or carbohydrate polyphosphonate (e.g., teichoic acid). Examples of synthetic anionic copolymers of methacrylic acid include a copolymerization product of an acrylic or methacrylic acid with a vinyl monomer including, for example, acrylamide, N,N-dimethyl acrylamide or N-vinylpyrrolidone. Exemplary anionic biopolymers or modified biopolymers include hyaluronic acid, glycosaminoglycanes such as heparin or chondroitin sulfate, fucoidan, poly-aspartic acid, poly-glutamic acid, carboxymethyl cellulose, carboxymethyl dextrans, alginates, pectins, gellan, carboxyalkyl chitins, carboxymethyl chitosans, and sulfated polysaccharides.

In some embodiments the nature of interaction and/or the density of surface modifiers on the particle can be adjusted to control the strength of the attractive interactions between the particles. In some embodiments these changes can be used to impact the physical properties of the resulting gel, i.e. the rigidity in non-shear or the fluidity in shear conditions, the overall mechanical strength of the gel, etc. Exemplary physical properties that can be modified include tensile strength, elongation, flexural strength, flexural modulus, viscosity under shear, etc.

D. Therapeutic, Prophylactic or Diagnostic Agents

The compositions described herein can be used for the responsive and/or controlled delivery of one or more therapeutic, prophylactic, or diagnostic agents. In some embodiments the compositions contain only a single therapeutic, prophylactic, or diagnostic agent, i.e. insulin. In other embodiments multiple agents can be delivered either in a responsive manner or in a controlled manner, either together or independently. For example, in some embodiments it can be advantageous to provide sustained extended release of a first therapeutic, prophylactic, or diagnostic agent, while at the same time providing for release of a second therapeutic, prophylactic, or diagnostic agent that is responsive to a particular stimulus. In such embodiments the first agent can be incorporated into polymeric particles containing a standard extended-release polymeric matrix as are known in the art. The second agent can be incorporated into polymeric particles containing a responsive polymeric matrix as described herein. For a specific, non-limiting example, in the treatment of diabetes it may be desirable to provide sustained delivery of a diabetes medication such as an alpha-glucosidase inhibitor while at the same time providing for delivery of insulin in response to detected increases in blood glucose levels. This is accomplished in some embodiments by providing network gel compositions containing both extended-release particles (i.e. PLGA particles) encapsulating the alpha-glucosidase inhibitor and glucose-responsive particles encapsulating insulin (such as the modified Dextran particles described in the examples herein).

In preferred embodiments compositions are provided containing insulin or an insulin analog. “Insulin” refers to a natural peptide hormone made by the pancreas that controls the level of the sugar glucose in the blood. Insulin permits cells to use glucose. “Insulin analog” as used herein refers to human insulin in which one or more amino acid residues have been replaced by another amino acid residue or deleted or in which the A chain and/or the B chain has been extended by addition of one or more amino acid residues at the N-terminal or at the C-terminal and which controls the level of glucose in the blood but with different pharmacokinetics than the naturally occurring insulin. Examples of insulin analogs include NPH insulin; also known as Humulin N, Novolin N, Novolin NPH, NPH Iletin II, and isophane insulin, marketed by Eli Lilly and Company under the name Humulin N, is an intermediate-acting insulin given to help manage the blood sugar level of those with diabetes. Many people reported problems following being switched to these insulins in the 80s, from porcine/bovine insulins. Problems included mood/character changes, memory problems, and hypo-unawareness.

Lispro.

Eli Lilly and Company had the first insulin analogue with “lispro” as a rapid acting insulin analogue. It is marketed under the trade name Humalog. It was engineered through recombinant DNA technology so that the penultimate lysine and proline residues on the C-terminal end of the B-chain were reversed. This modification did not alter the insulin receptor binding, but blocked the formation of insulin dimers and hexamers. This allowed larger amounts of active monomeric insulin to be available for postprandial (after meal) injections.

Aspart.

Novo Nordisk created “aspart” and marketed it as NovoLog/NovoRapid (UK-CAN) as a rapid acting insulin analogue. It was created through recombinant DNA technology so that the amino acid, B28, which is normally proline, is substituted with an aspartic acid residue. The sequence was inserted into the yeast genome, and the yeast expressed the insulin analogue, which was then harvested from a bioreactor. This analogue also prevents the formation of hexamers, to create a faster acting insulin. It is approved for use in CSII pumps and Flexpen, Novopen delivery devices for subcutaneous injection.

Glulisine.

Glulisine is a newer rapid acting insulin analog from Sanofi-Aventis, approved for use with a regular syringe, in an insulin pump or the Opticlik Pen. Standard syringe delivery is also an option. It is sold under the name Apidra. The FDA-approved label states that it differs from regular human insulin by its rapid onset and shorter duration of action.

Shifted Isoelectric Point Insulins

Normal unmodified insulin is soluble at physiological pH. Analogues have been created that have a shifted isoelectric point so that they exist in a solubility equilibrium in which most precipitates out but slowly dissolves in the bloodstream and is eventually excreted by the kidneys. These insulin analogues are used to replace the basal level of insulin, and may be effective over a period of up to 24 hours. However, some insulin analogues, such as insulin detemir, bind to albumin rather than fat like earlier insulin varieties, and results from long-term usage (e.g. more than 10 years) have never been released.

Glargine Insulin.

Sanofi-Aventis developed glargine as a longer lasting insulin analogue, and markets it under the trade name Lantus. It was created by modifying three amino acids. Two positively charged arginine molecules were added to the C-terminus of the B-chain, and they shift the isoelectric point from 5.4 to 6.7, making glargine more soluble at a slightly acidic pH and less soluble at a physiological pH. Replacing the acid-sensitive asparagine at position 21 in the A-chain by glycine is needed to avoid deamination and dimerization of the arginine residue. These three structural changes and formulation with zinc result in a prolonged action when compared with biosynthetic human insulin. When the pH 4.0 solution is injected, most of the material precipitates and is not bioavailable. A small amount is immediately available for use, and the remainder is sequestered in subcutaneous tissue. As the glargine is used, small amounts of the precipitated material will move into solution in the bloodstream, and the basal level of insulin will be maintained up to 24 hours. The onset of action of subcutaneous insulin glargine is somewhat slower than NPH human insulin. It is clear solution as there is no zinc in formula.

Detemir Insulin.

Novo Nordisk created insulin detemir and markets it under the trade name Levemir as a long-lasting insulin analogue for maintaining the basal level of insulin. The basal level of insulin may be maintained for up to 20 hours, but the time is clearly affected by the size of the injected dose. This insulin has a high affinity for serum albumin, increasing its duration of action.

Diabetes Medications

Exemplary diabetes medications include sulfonylureas, meglitinides, biguanides, thiazolidinediones, alpha-glucosidase inhibitors, or DPP-4 inhibitors. Sulfonylureas stimulate the beta cells of the pancreas to release more insulin. Chlorpropamide (Diabinese) is the only first-generation sulfonylurea still in use today. The second generation sulfonylureas are used in smaller doses than the first-generation drugs. There are three second-generation drugs: glipizide (Glucotrol and Glucotrol XL), glyburide (Micronase, Glynase, and Diabeta), and glimepiride (Amaryl). Meglitinides are drugs that also stimulate the beta cells to release insulin. Repaglinide (Prandin) and nateglinide (Starlix) are meglitinides. Metformin (Glucophage) is a biguanide. Biguanides lower blood glucose levels primarily by decreasing the amount of glucose produced by the liver. Rosiglitazone (Avandia) and pioglitazone (ACTOS) are in a group of drugs called thiazolidinediones. These drugs help insulin work better in the muscle and fat and also reduce glucose production in the liver. DPP-4 inhibitors help improve A1C without causing hypoglycemia. They work by preventing the breakdown of a naturally occurring compound in the body, GLP-1. GLP-1 reduces blood glucose levels in the body, but is broken down very quickly so it does not work well when injected as a drug itself. By interfering in the process that breaks down GLP-1, DPP-4 inhibitors allow it to remain active in the body longer, lowering blood glucose levels only when they are elevated. Sitagliptin (JANUVIA) and saxagliptin (ONGLYZA) are the two DPP-4 inhibitors currently on the market.

In certain preferred embodiments compositions are provided containing insulin and one or more additional diabetes medications that can be delivered together in a responsive manner, or independently by providing extended release of the diabetes medication in combination with responsive release of the insulin or insulin analog in response to increased glucose levels.

In addition to insulin and insulin analogs, other therapeutic, prophylactic or diagnostic agents can be encapsulated to treat or manage diseases or disorders. These can include small drugs, proteins or peptide, nucleic acid molecules such as DNA, mRNA and siRNA, polysaccharides, lipids, and combinations thereof

The specific therapeutic, prophylactic, or diagnostic agents encapsulated will depend upon the condition to be treated. For example, in compositions containing a polymeric matrix responsive to blood alcohol levels it may be advantageous to use one or more drugs commonly used for treating alcoholism or other addictions, i.e. disulfiram or calcium carbamide, diazepam or librium, or an opiate antagonists such as naloxone, naltrexone, cyclazocine, diprenorphine, etazocine, levalorphan, metazocine, or nalorphine.

Diagnostic agents may be release alone or in combination with therapeutic and/or prophylactic agents. Examples include radionuclides, radiopaque molecules, and MRI, x-ray or ultrasound detectable molecules.

E. Excipients

“Pharmaceutically acceptable carrier” as used herein means a non-toxic, inert solid, semi-solid or liquid filler, diluent, encapsulating material or formulation auxiliary of any type. Remington's Pharmaceutical Sciences Ed. by Gennaro, Mack Publishing, Easton, Pa., 1995 discloses various carriers used in formulating pharmaceutical compositions and known techniques for the preparation thereof.

The formulation may consist solely of the particles and resulting gel encapsulating the insulin or insulin analog and glucose-responsive components, or include one or more excipients such as sterile water, sterile buffered saline, or other standard excipient.

### II. Methods of Making Responsive Compositions

A. Methods of Making Nanonetwork Gels

Colloidal nanonetwork gels can be fabricated using oppositely-charged particles, such as nanoparticles or microparticles, which interact to form stable three-dimensional scaffolds. That is, the colloid gels can be molded and/or shaped into responsive drug delivery scaffolds for a variety of uses. The shaping can be done prior to implantation to form a stable structure or can be done during implantation so as to form the stable structure in situ. The scaffolds can be configured with a desired degree of malleability under shear and strong static cohesion so as to facilitate fabrication of shape-specific tissue scaffolds. Also, a charged polymer can be substituted for one of the charged particles during the manufacture process to produce a colloid gel having a charged particle and an oppositely charged polymer. As such, the descriptions herein can include one charged particle being substituted with a charged polymer.

The colloid gels can be prepared from biodegradable particles and/or biostable particles. At least one of the particles contains a responsive drug delivery polymer matrix and a therapeutic, prophylactic, or diagnostic agent to be delivered in a responsive manner, i.e. in response to a change in one or more conditions. As such, at least some of the particles will preferably be polymeric. Additional particles can be polymeric, organic, inorganic, ceramic, minerals, combinations thereof, and the like. The colloid gels can include more than one type of particle, such as a biodegradable polymer and a mineral.

Colloidal gels can be prepared from oppositely-charged nanoparticles at high concentration exhibit pseudoplastic behavior that allows for the fabrication of shape-specific microscale materials. The cohesive strength of these materials depends upon interparticle interactions such as; electrostatic forces, van der Waals attraction, steric hindrance, and the like which may be leveraged to facilitate the design of nanonetwork gels having the desired mechanical and physical properties in addition to the desired release of therapeutic, prophylactic, or diagnostic agents.

FIGS. 1A-1C provides a schematic representation of one process for preparing a network gel that can be formed and/or molded into an implant for use as a therapeutic scaffold. As shown in FIG. 1A, positive chitosan coated particles and negative alginate coated particles can be combined to prepare a porous colloid gel (FIG. 1C). In some embodiments, the colloid gel can then be molded into an implant that can have any of a variety of shapes. Often, the shapes will be in a form suitable for implantation. The implant can then be implanted to provide a therapeutic scaffold for responsive delivery of one or more therapeutic, prophylactic, or diagnostic agents. In some embodiments the network gel implant will have a viscosity under shear conditions to allow injection with a standard needle. In a preferred embodiment the implant provides delivery of insulin or an insulin analogue in response to blood glucose levels (FIG. 1C).

In some embodiments, positive particles and negative particles can be combined to prepare a porous colloid gel, which is a network framed with particles. The colloid gel is substantially as described herein and includes a network of positive particles that are associated with negative particles in order to form a matrix with pores in the form of a colloidal network gel. The colloidal gel has a shear-thinning characteristic in that when a shear force is applied to the colloid gel, such as from being injected from a syringe, passed through a tube, or being stirred, the positive particles and negative particles can become disassociated so as to form a paste or to provide some increased fluidity to the colloid gel. Accordingly, the particle network can be temporarily destroyed to provide the fluidity. The fluidity can be similar to that of a paste such that the colloidal gel is moldable and can be shaped with a spatula or other utensil. When under no shear force, the positive particles and negative particles can again be combined to form the porous colloidal network gel. The colloid gel can then set up into a structurally sound form when no shear is applied. Thus, the set up colloid gel can be used as an implant and can be injected into a site within a body to provide a moldable and shapeable implant in situ.

The colloidal gel scaffold can be used for prolonged delivery of one or more therapeutic, prophylactic, or diagnostic agents, and can include a first plurality of positively-charged biocompatible particles and a second plurality of negatively-charged biocompatible particles. The positive and negative particles can be linked together through ionic interactions or other interactions so as to form a three-dimensional matrix in the form of a colloid gel. Optionally, the matrix can include a plurality of pores defined by and disposed between the particles. The pores can be smaller than the particles or sized sufficient for receiving a plurality of living cells. For example, the pores can be the interstitial space between the particles or larger pores. Accordingly, the pores can be dimensioned to retain small molecules, macromolecules, cells, and the like. Also, the linked particles can have a surface area sufficient for interacting with the cells within the plurality of pores and on the scaffold prepared from the particles.

The biocompatible particles can include first and second sets of particles. Generally, the first set of particles is positively charged and the second set of particles is negatively charged, or vice versa. Additionally, the first set of particles can have a first characteristic other than charge type. The second set of particles can have a second characteristic other than charge type that is different from the first characteristic. For example, the first and second characteristics can be independently selected from the following: composition; polymer; particle size; particle size distribution; zeta potential; charge density; type of bioactive agent; type of bioactive agent combination; bioactive agent concentration; amount of bioactive agent; rate of bioactive agent release; mechanical strength; flexibility; rigidity; color; radiotranslucency; or radiopaqueness.

The oppositely-charged particles can be combined into a comingled spatial distribution such that positive particles are associated with negative particles to form a matrix. In some instances, a portion of the matrix can have more particles with one type of charge than the other, and the other type of particles can have a higher charge density. That is, more particles with a lower charge density can be combined with fewer particles with a higher charge density in order to form the colloid gel matrix.

In one embodiment, a colloid gel for use as responsive drug delivery scaffold can be prepared by substituting only one of the particles with a polymer. This can include a plurality of positive charged polymers being combined with a plurality of negative charged particles, or a plurality of negative charged polymers being combined with a plurality of positive charged particles. The charged polymer can have various molecular weights; however, larger and/or longer polymers can be useful and more particle like.

The polymer can be branched, crosslinked, or linear. The charged polymer can include a charge density similar to the particles. Also, the polymer can have a plurality of units that carry the charge. The polymer can be substituted for either a positive particle or a negative particle, and a particle of opposite charge of the polymer can be combined therewith in order to prepare a colloid gel having the properties described herein for use as a responsive drug delivery scaffold.

The process of making the responsive drug delivery scaffolds with oppositely charged particles successfully produces porous, well-connected matrices, which may be suitable for a variety of applications depending on the selection of suitable therapeutic, prophylactic, or diagnostic agents. The process can be used to create porous, biocompatible and biodegradable scaffolds using particles made of, for example, poly(D,L-lactide-co-glycolide) (PLG), poly(D,L-lactic-co-glycolic acid) (PLGA). Additionally, porosity patterns can be created within a scaffold using particles of different sizes.

In some embodiments, a first set of particles having a positive charge is provided; a second set of particles having a negative charge with one or both sets containing a responsive polymeric matrix and a therapeutic, prophylactic, or diagnostic agent to be delivered are provided; and the particles of the first set and second set are combined to form a three-dimensional matrix having a plurality of pores defined by and disposed between the particles. The plurality of particles have a surface area sufficient to allow cells to pass within the plurality of pores. The three-dimensional matrix can include the first set and second set of particles comingled such that the positive particles are adjacent and ionically associated with the negative particles so as to form the matrix.

The scaffolds can be fabricated by flowing oppositely charged particle suspensions into a mold of pre-determined shape (to allow fabrication of shape-specific materials) with predefined flow profiles. The oppositely charged particles can be combined and mixed together so as to associate and form a continuous material. The process can utilize commercially available programmable syringe pumps (e.g., Motor-driven syringe pumps) to pump the oppositely charged particles into a mold. These types of pumps can now be used with oppositely charged particle compositions to create three-dimensional drug delivery scaffolds with various characteristics.

In some embodiments freeform printing of the oppositely charged particle compositions can be used to form colloidal gels that can be shaped by printing, molding, or cutting, to produce three-dimensional microperiodic networks exhibiting precise structure. Additionally, the colloid gels can be molded and freeze dried to create more rigid structures or directly injected as in situ forming scaffolds. Application of porogens, such as sodium chloride, salts, oil, parafins, polymers, or surfactants, to the scaffolds can create pores of various sizes to enhance interconnected pore 3-D structure.

In some embodiments, the method of preparing a particle-based scaffold can include any one of the following: preparing a first liquid suspension of the first set of positive particles; preparing a second liquid suspension of the second set of negative particles with either the first set, second set, or both sets containing a responsive polymeric matrix and a therapeutic, prophylactic, or diagnostic agent; introducing the first liquid suspension into a mold; introducing the second liquid suspension into the mold before, during, and/or after introducing the first liquid suspension into the mold; molding the first and second set of particles into a mold with the positive charges associating with the negative charges so as to form a matrix.

In some embodiments, the first and second particles can be combined, and then introduced into a body of a subject to form the matrix. The matrix can then be shaped as needed or desired. For example, the first particle composition can be combined with the second particle composition, and the combined composition can be deposited into a desired location within the body of a subject. Thus, the composition can be pre-shaped prior to implantation or shaped after being deposited within a body of a subject. In one embodiment, the particles can include immobilized surface factors (e.g., RGD adhesion sequences). A distribution of particles having immobilized surface factors that produce a gradient of such factors can influence cell migration.

The responsive drug delivery scaffolds can be made from oppositely charged monodispersed particles, which may lead to improved systems to explore the effects of particle size and charge density on particle-based scaffolds. Uniform particles can pack closely compared to randomly-sized particles, providing better control over the pore-sizes and porosity of the scaffold, and may considerably aid the mechanical integrity of the scaffolds. Moreover, local release of molecules from the particles in a bulk scaffold is related to individual particle size and polymer properties. Reproducibility and predictability associated with uniform particle-based scaffolds may make them more suitable for a drug delivery scaffold. Various charge densities can also be used in a single scaffold.

In some embodiments, by using two or more different polymers (e.g., a copolymer such as a diblock copolymer and a homopolymer) properties of particles can be controlled. In some embodiments the methods described herein form nanoparticles that have a high amount of encapsulated therapeutic agent, for example, that may include about 0.2 to about 40 weight percent, or about 0.2 to about 30 weight percent, e.g., about 0.2 to about 20 weight percent or about 1 to about 10 weight percent of a therapeutic, prophylactic, or diagnostic agent.

In some embodiments, a nanoemulsion process is used to encapsulate a therapeutic, prophylactic, or diagnostic agent, with a first polymer (for example, PLA-PEG or PLGA-PEG) and/or a second polymer (modified polysaccharides such as dextrsoe), is mixed with an organic solution to form a first organic phase. Such first phase may include about 5 to about 50% weight solids, e.g. about 5 to about 40% solids, or about 10 to about 30% solids, e.g. about 10%, 15%, 20% solids. The first organic phase may be combined with a first aqueous solution to form a second phase. The organic solution can include, for example, acetonitrile, tetrahydrofuran, ethyl acetate, isopropyl alcohol, isopropyl acetate, dimethylformamide, methylene chloride, dichloromethane, chloroform, acetone, benzyl alcohol, TWEEN® 80, or Span 80, and combinations thereof. In some embodiments, the organic phase may include benzyl alcohol, ethyl acetate, and combinations thereof. The second phase can be between about 1 and 50 weight %, e.g., 5-40 weight %, solids. The aqueous solution can be water, optionally in combination with one or more of sodium cholate, ethyl acetate, and benzyl alcohol.

As an example, the oil or organic phase may use solvent that is only partially miscible with the nonsolvent (water). Therefore, when mixed at a low enough ratio and/or when using water pre-saturated with the organic solvents, the oil phase remains liquid. The oil phase may be emulsified into an aqueous solution and, as liquid droplets, sheared into nanoparticles using, for example, high energy dispersion systems, such as homogenizers or sonicators. The aqueous portion of the emulsion, otherwise known as the “water phase”, may be surfactant solution consisting of sodium cholate and pre-saturated with ethyl acetate and benzyl alcohol.

Emulsifying the second phase to form an emulsion phase may be performed in one or two emulsification steps. For example, a primary emulsion may be prepared, and then emulsified to form a fine emulsion. The primary emulsion can be formed, for example, using simple mixing, a high pressure homogenizer, probe sonicator, stir bar, or a rotor stator homogenizer. The primary emulsion may be formed into a fine emulsion through the use of e.g. probe sonicator or a high pressure homogenizê e.g. by using 1, 2, 3 or more passes through a homogenizer. For example, when a high pressure homogenizer is used, the pressure used may be about 5000 to about 15000 psi, or about 9900 to about 13200 psi, e.g. 9900 or 13200 psi.

Either solvent evaporation or dilution may be needed to complete the extraction of the solvent and solidify the particles. For better control over the kinetics of extraction and a more scalable process, a solvent dilution via aqueous quench may be used. For example, the emulsion can be diluted into cold water to a concentration sufficient to dissolve all of the organic solvent to form a quenched phase. Quenching may be performed at least partially at a temperature of about 5° C. or less. For example, water used in the quenching may be at a temperature that is less that room temperature {e.g. about 0 to about 10° C., or about 0 to about 5° C.).

B. Incorporation of Therapeutic, Prophylactic or Diagnostic

The particles of the colloidal gel matrix will include at least one therapeutic, prophylactic, or diagnostic agent that is released in response to a stimulus or condition, preferably insulin that is release in response to increase blood glucose levels. The colloid gel matrix can also include additional therapeutic, prophylactic, or diagnostic agents contained in or disposed on a first set of particles or either charge. The therapeutic, prophylactic, or diagnostic agents can also be disposed in the interstitial spaces between the linked particles. The resulting scaffold can be configured to release the additional therapeutic, prophylactic, or diagnostic agents so as to create a desired concentration of the agent. Optionally, a second set of particles can be substantially devoid of the agent, or can include a second agent. When the second agent is contained in or disposed on the second set of particles, the scaffold can be configured to release the second agent so as to create a desired concentration of the second agent that is the same or different from the first desired concentration of the first agent. The different agents can be in both positive and negative particles or in distinct particles and can be released in a controlled and/or responsive fashion. For example, the positive particles can include a first agent and the negative particles can include a second agent. Also, the positive particles can include more than one type of agent. Moreover, the same therapeutic, prophylactic, or diagnostic agent can be in both positive and negative particles. This allows for a diverse and complex configuration of particles so that desired release profiles of one or more therapeutic prophylactic, or diagnostic agents can be obtained and can be responsive to one or more stimuli such as changes in concentration of one or more molecules such as blood glucose, changes in pH, or changes in temperature. Furthermore, particles with one type of agent can be preferentially disposed on one side of the colloid gel matrix with a different type of agent in a different side or portion of the matrix. The configuration of different particles with different agents can be achieved during the manufacturing process by locating one type of particle in one position within a mold and a different type of particle in a different position. Thus, a number of different types of particles can each have a bioactive agent to provide a plurality of different types of therapeutic, prophylactic, or diagnostic agents to the scaffold.

Accordingly, the first characteristic of a first set of particles can be a first agent contained in or disposed on the particles, and the second characteristic of a second set of particles can be a second agent contained in or disposed on the particles. In some embodiments, at least one of a first set or second set of particles can include a biodegradable polymer. For example, the particles can include a poly-lactide-co-glycolide or poly(lactic-co-glycolic acid) or PLGA or other similar polymer or copolymer.

In one embodiment, the scaffold can include a third set of particles having a third characteristic other than charge that is the same or different from the first or second characteristics. The third set of particles can have a predetermined spatial location that is different from or the same as the spatial locations of the positive and negative particles with respect to the matrix. Also, the third set can be positive, negative, or neutral. When neutral, the particles can be entrapped within a matrix of positive/negative particles or can be chemically bound thereto.

In one embodiment, the scaffold can include a first end and an opposite second end. Accordingly, a first set of particles can have a first therapeutic, prophylactic, or diagnostic agent, and the first end can have a majority of particles of the first set. Correspondingly, a second set of particles can have a second therapeutic, prophylactic, or diagnostic agent that is different from the first agent, and the second end having a majority of particles of the second set. The positive and negative surface charges on the particles interact to form a gel.

Methods of making drug loaded particles are known in the art. In some embodiments the polymeric particles are loaded with one or more therapeutic, prophylactic, or diagnostic agents prior to forming the gel. In some embodiment one or more therapeutic, prophylactic, or diagnostic agents is loaded into the particles after the gel has formed, for instance by soaking the gel in a solution containing the agent. In preferred embodiments the agent is insulin or an insulin analog. Insulin is loaded into the hydrogel before or after mixing the components which self-assemble into a hydrogel. In some embodiments insulin loaded particles are first created having a responsive polymeric matrix, optionally having a surface modifier, and the particles are combined to form the gel. In one embodiment, a hydrogel that is already formed and contains the responsive polymeric particles is incubated with an insulin solution to absorb the insulin.

In some embodiments, not all of the therapeutic, prophylactic, or diagnostic agent is encapsulated in the particles at this stage, and a drug solubilizer is added to the quenched phase to form a solubilized phase. The drug solubilizer may be for example, TWEEN® 80, TWEEN® 20, polyvinyl pyrrolidone, cyclodextran, sodium dodecyl sulfate, or sodium cholate. For example, TWEEN® 80 may be added to the quenched nanoparticle suspension to solubilize the free drug and prevent the formation of drug crystals. In some embodiments, a ratio of drug solubilizer to therapeutic agent is about 100:1 to about 10:1.

The solubilized phase may be filtered to recover the nanoparticles. For example, ultrafiltration membranes may be used to concentrate the nanoparticle suspension and substantially eliminate organic solvent, free drug, and other processing aids (surfactants).

Exemplary filtration may be performed using a tangential flow filtration system. For example, by using a membrane with a pore size suitable to retain nanoparticles while allowing solutes, micelles, and organic solvent to pass, nanoparticles can be selectively separated. Exemplary membranes with molecular weight cut-offs of about 300-500 kDa (−5-25 nm) may be used.

Diafiltration may be performed using a constant volume approach, meaning the diafiltrate (cold deionized water, e.g. about 0° C. to about 5° C., or 0 to about 10° C.) may added to the feed suspension at the same rate as the filtrate is removed from the suspension. In some embodiments, filtering may include a first filtering using a first temperature of about 0° C. to about 5° C., or 0° C. to about 10° C., and optionally a second temperature of about 20° C. to about 30° C., or 15° C. to about 35° C. For example, filtering may include processing about 10 to about 20 diavolumes at about 0° C. to about 5° C. In another embodiment, filtering may include processing about 1 to about 6 diavolumes at about 0° C. to about 5° C., and processing at least one diavolume (e.g. about 1 to about 3 or about 1-2 diavolumes) at about 20° C. to about 30° C.

Optionally, after purifying and concentrating the nanoparticle suspension, the particles may be passed through one, two or more sterilizing and/or depth filters, for example, using −0.2 μπl depth pre-filter.

In an exemplary embodiment of preparing nanoparticles, an organic phase is formed composed of a mixture of a therapeutic agent, e.g., insulin, and polymer (homopolymer, and co-polymer). The organic phase may be mixed with an aqueous phase at approximately a 1:5 ratio (oil phase:aqueous phase) where the aqueous phase is composed of a surfactant and optionally dissolved solvent. A primary emulsion may then formed by the combination of the two phases under simple mixing or through the use of a rotor stator homogenizer. The primary emulsion is then formed into a fine emulsion through the use of e.g. high pressure homogenizer. Such fine emulsion may then quenched by, e.g. addition to deionized water under mixing. An exemplary quench:emulsion ratio may be about approximately 8:1. A solution of TWEEN® (e.g., TWEEN® 80) can then be added to the quench to achieve e.g. approximately 1-2% TWEEN® overall, which may serve to dissolve free, unencapsulated insulin. Formed nanoparticles may then be isolated through either centrifugation or ultrafiltration/diafiltration.

C. Dosage Forms

Dosage forms may be formulated in a conventional manner using one or more physiologically acceptable carriers comprising known excipients and auxiliaries which facilitate processing into preparations which can be used pharmaceutically. In one embodiment, prior to injection, the formulation is in the form of a suspension.

Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). Proper formulation is dependent upon the route of administration chosen.

In a preferred embodiment, the formulation is an injectable formulation. An injectable insulin formulation can be made by suspending the gel-encapsulated insulin in a diluent. The suspension is sterilized and filled in a vial suitable for unit or multiple injection dosing. Sterile injectable preparations may be formulated as known in the art. Among the acceptable vehicles and solvents that may be employed are water, Ringer's solution, U.S.P. and isotonic sodium chloride solution. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

### IV. Methods of Using Compositions

A. Methods of Administration

The formulations can be administered subcutanteously, intramuscularly, or intradermally. In preferred embodiment, the formulation is injected subcutaneously. In some embodiments the gel is preformed and injectable under shear conditions. In such embodiments it can be possible to inject the gel using standard needles. In some embodiments the gel is formed in situ, for example by combining a first and second plurality of particles in situ to form the gel or by combining particles where the gel is formed upon the application of heat, light such as UV, or some other external stimulus to form the gel. For example, particles coated with a thermally activated crosslinker can be gelled in situ by the application of heat or, in some embodiments, simply by the increase to physiological temperatures.

“Dosage unit form” as used herein refers to a physically discrete unit of conjugate appropriate for the patient to be treated. In one embodiment, the formulation is an insulin formulation designed to release insulin into systemic circulation over time with a basal release profile following injection in a patient. In another embodiment, the formulation is designed to release insulin into systemic circulation over time with a non-basal release profile following injection in a patient. Exemplary non-basal release profiles include a regular human insulin release profile and a prandial release profile.

In one embodiment the formulation is designed to release insulin into systemic circulation over time with a regular human insulin release profile following injection in a patient. In another embodiment, the formulation is designed to release insulin into the systemic circulation over time with a prandial release profile following injection in a patient.

B. Patients to be Treated

The compositions and formulations including a responsive drug delivery scaffold or gel can be administered to a patient in need of delivery of a therapeutic, prophylactic, or diagnostic agent in a responsive manner. In a preferred set of embodiments, the patient is in need of administration of a therapeutic agent in response to increases in blood glucose levels, i.e. due to diabetes.

In some embodiments, as the patient's blood glucose levels rise, the glucose is converted into gluconic acid by the responsive signaling means (for example the GOx/CAT system encapsulated in the particles). The gluconic acid locally lowers the pH resulting in an increased degradation of the acid degradable polymeric matrix (such as the modified dextrose polymers described herein although other polymers can be used). Upon degradation the particles release the encapsulated insulin which, because the rate is dependent upon the conversion of blood glucose into gluconic acid, can be delivered in a glucose-dependent manner.

In some embodiments, as the patient's blood glucose levels rise, the glucose binds to the glucose binding porting of the peptide hydrogel, disrupting interactions contributing to gel formation. The interaction with glucose leads to release from the insulin formulation in a blood glucose-dependent manner as shown in FIG. 1C.

In some embodiments, the insulin formulation is administered to patients who are not fully insulin dependent. In one embodiment, the formulation provides a sufficient amount of insulin to the patient during the day so that the patient does not require additional insulin-containing formulations to maintain his/her blood glucose levels within a safe range. The patient is typically not fully insulin dependent.

In another embodiment, the formulation is administered to a patient who is receiving intensive insulin therapy as one of the insulin-containing formulations administered to the patient during the day. Preferably the formulation delivers insulin to the patient with a basal release profile.

As used herein, “controlling blood glucose levels” refers to the maintenance of blood glucose concentrations at a desired level, typically between 70-130 mg/dL or 90-110 mg/dL.

In preferred embodiments, the formulation when administered to a patient with diabetes is able to maintain normoglycemia (normal glycemic levels) for a period of up to 5 days, 1 week, 2 weeks, one month, or up to two months.

The present invention will be further understood by reference to the following non-limiting examples.

### EXAMPLES

All chemicals were purchased from Sigma-Aldrich unless otherwise specified, and were used as received. Human recombinant insulin (Zn salt, 27.5 IU/mg) was purchased from Invitrogen. The deionized water was prepared by a Millipore NanoPure purification system (resistivity higher than 18.2 MΩ·cm-1).

### Example 1

### Preparation of m-Dextran

Briefly, 1.0 g of dextran (Mn˜9-11 kDa) was added to a flame-dried round bottom flask and purged with nitrogen. 10 mL of anhydrous dimethyl sulfoxide was added to the flask and stirred until complete dissolution of the dextran. Pyridinium p-toluenesulfonate (PPTS, 15.6 mg, 0.062 mmol) was added to the solution followed by the addition of 2-ethoxypropene (4.16 mL, 37 mmol). The reaction mixture was purged with nitrogen briefly, and then sealed with parafilm to prevent reactant evaporation. The reaction was stirred at room temperature for 30 minutes resulting in m-dextran. At that point, the reaction was quenched by the addition of 1 mL of triethylamine. The mixture was then precipitated and washed three times in basic water (pH=˜8) to prevent undesired degradation and collected by centrifugation (8000 rpm, 15 min). The product was lyophilized to remove residual water and resulted in a white solid. IR (KBr, cm-1): 3485, 2971, 2862, 1471, 1374, 1246, 1203, 1047, 806, 746, 549. 1H NMR (400 MHz, CDCl3): 5.21 (br, 1H), 4.87 (br, 1H), 4.10-3.70 (br, 12H), 3.46 (br, 4H), 1.30 (br, 16H), 1.11 (br, 8H).

### Example 2

### Preparation of Glucose-Responsive Nanoparticles and Nano-Network Gels

Materials and Methods

Dextran nanoparticles were prepared by an improved double-emulsion (water-in-oil-in-water) solvent evaporation/extraction method. Briefly, 5.8 mL organic phase (dichloromethane (DCM)) containing 240 mg m-dextran was emulsified with 0.5 mL aqueous phase containing 35 mg human recombinant insulin (Invitrogen, USA) only or together with 3.5 mg enzymes (weight ratio of glucose oxidase to catalase: 4:1) by sonication for 45 cycles (1 sec each with a duty cycle of 60%). Thereafter, the primary emulsion was immediately poured into 25 mL of the chitosan or alginate aqueous solution (1%) and sonicated for 45 cycles. The double emulsion was subsequently transferred into 150 mL of chitosan (Mn: 612 kDa; degree of deacetylation: 96.1%) or alginate (Mw: 1.6×10⁵) aqueous solution (0.2%). The mixed suspension was stirred at room temperature to eliminate

DCM by evaporation. After 2 hours, the resulting nanoparticles were cleaned and collected by repeating a procedure of centrifuging at 10,000 rpm and resuspending in distilled water three times. The product was dried by lyophilization and stored at 4° C. To obtain nano-network gels, chitosan or alginate coated nanoparticles were separately dispersed in deionized water (w/v=20%) and mixed together (w/w=1/1) in a bath sonitator for 2 min. The prepared nano-network gel was collected by centrifuging at 3000 rpm for 3 min and stored at 4° C.

The loading capacity (LC) and encapsulation efficiency (EE) of insulin encapsulated nanoparticles were determined by measuring the amount of non-encapsulated insulin through BCA (bicinchoninic acid) protein assay and using insulin-free particles as basic correction. LC and EE were calculated as: LC=(A-B)/C, EE=(A-B)/A, where A was expected encapsulated amount of insulin, B was the free amount of insulin in the collection solution and C was the total weight of particles.

Results

In order to obtain oppositely-charged nanoparticles, chitosan and alginate, were employed as surface modifiers to coat dextran nanoparticles. Both chitosan and alginate were exploited to introduce both a positive and negative, respectively, surface charge on dextran nanoparticles. Utilizing a double emulsion procedure, two types of dextran particles were prepared separately with an insulin loading capacity of 7.9±0.8 wt % for the chitosan-coated particles and 11.4±0.1 wt % for the alginate-coated particles. As shown by scanning electron microscopy (SEM), both chitosan and alginate coated nanoparticles have nearly monodisperse particle sizes. The average hydrodynamic particle sizes determined by dynamic light scattering (DLS) for chitosan-coated and alginate-coated particles were 340 nm and 293 nm, respectively. The zeta potentials for both particles were 10.6±1.9 mV (chitosan coated) and −11.5±1.7 mV (alginate coated), respectively. The uniform and small size of these nanoparticles provided the cohesive strength while the opposite zeta potentials promoted tight particle packing as a result of the electrostatic interaction.

The cohesive nano-network loaded with both enzymes and insulin (designated NN(E+1)) was created by mixing the oppositely charged dextran nanoparticle solutions. SEM illustrates that nanoparticles were joined together to form a bulk porous structure comprised of a 3D network with microchannels. The formation of domains consisting of tightly packed agglomerates indicates that the cohesive nature of the nano-network resulted from an equilibrium of interparticle attractions (agglomerates) and repulsions (pores).

To further validate the interaction between oppositely charged particles, insulin conjugated with two different fluorescent dyes was encapsulated into nanoparticles coated with chitosan and alginate. 3D laser scanning confocal microscopy (LSCM) indicates that particles in the resulting network were compacted without noticeable mobility.

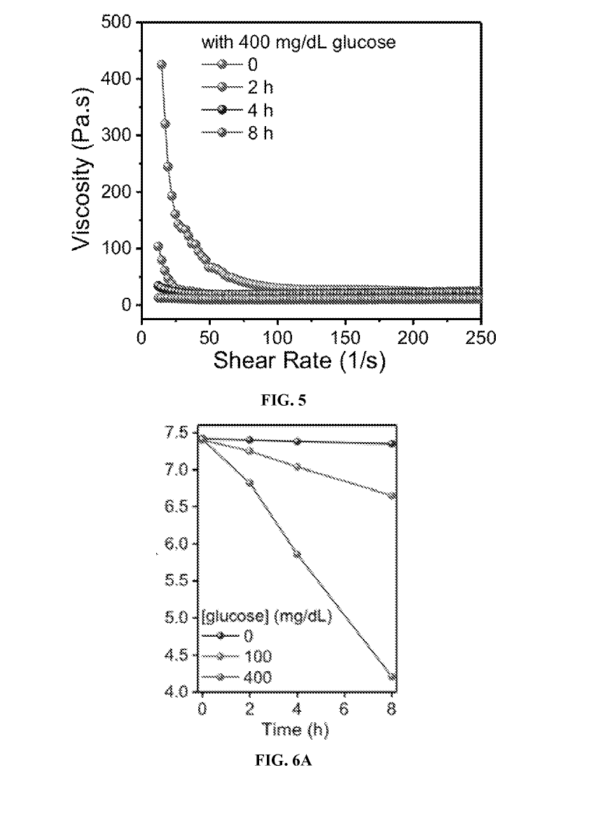

To investigate the injectability of the nano-network, the viscosities of each nanoparticle formulation and resulting nano-network with the same solid content were measured as a function of shear rate (FIG. 5). The initial viscosity of nano-network at low shear rates compared to pure nanoparticles confirmed the formation of attractive electrostatic interaction and compact nanoparticle packing. The cohesive forces were reduced at high shear rate resulting in the low viscosities that allows for convenient injection of the nano-network through syringes.

### Example 3

### In Vitro Release Studies

Materials and Methods

To examine the glucose-responsive dissociation of the nano-network, gels were collected in microcentrifuge tubes and incubated with PBS solutions in the absence or presence of glucose, both at a hyperglycemic level (400 mg dL-1) and a normal level (100 mg dL-1).

After preparation of the nano-network, various solutions (PBS, 100 mg dL-1 or 400 mg dL-1 glucose, 500 μL) were added to the each tube and incubated at 37° C. on an orbital shaker to evaluate the release of insulin. At predetermined time points, the sample was centrifuged (8000 rpm, 30 sec) and 12 μL of the supernatant was removed for analysis. 12 μL of fresh solution was then added to the tube to maintain a constant volume and placed back within the incubator. Total insulin content was measured using a Coomassie Plus Protein Assay. The absorbance of the well was detected at 595 nm and the concentration was interpolated from an insulin standard curve and a calibration curve using nano-network with enzymes only. To evaluate the material's ability to adapt to cyclical changes in glucose levels, each nano-network sample was first incubated in 400 mg dL-1 glucose (500 μL) for 2 hours at 37° C. At that point, the sample was centrifuged (8000 rpm, 30 sec) and all of the supernatant was recovered. The sample was washed twice with PBS and then incubated in 100 mg dL-1 glucose (500 μL) for another 2 hours. This cycle was repeated numerous times. Similarly, insulin concentration was determined using the Coomassie Plus Protein Assay.

Results

The nano-network material exposed to the hyperglycemic solution gradually dissociated over time. After 8 hours, the incubated solution became transparent due to the thorough hydrolysis of the modified dextran into native dextran. Meanwhile, the recorded pH values of incubating solutions substantiated that the degradation of network correlated to a decrease of the solution pH from 7.4 to 4.2, confirming the enzymatic conversion of glucose to gluconic acid. In contrast, both of the control samples (no glucose and 100 mg dL-1 glucose) did not display observable dissociation over the 8 hour time course, consistent with lack of change in the solution pH (FIG. 3). Additionally, the viscosity of the nano-network at low shear rates steadily decreased when exposed to hyperglycemic conditions (FIG. 5), as a result of decreasing cohesive forces due to the changing charges of polyelectrolytes as the pH decreases. The overall conformational structure of released insulin from the nano-network was maintained to that of native insulin demonstrated by the circular dichroism (CD) spectrum.

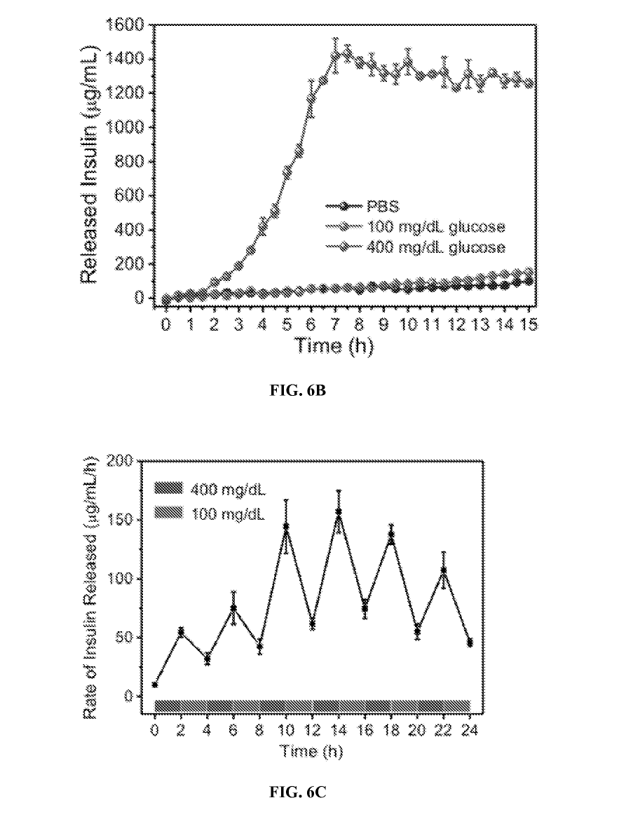

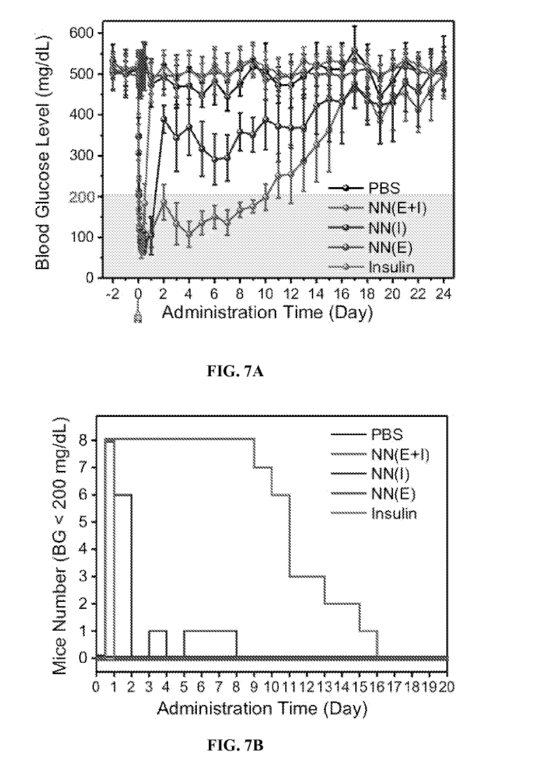

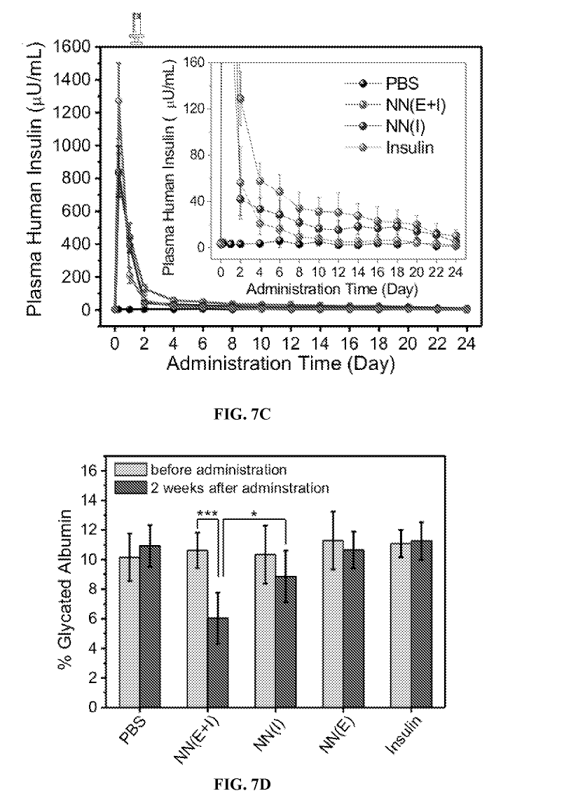

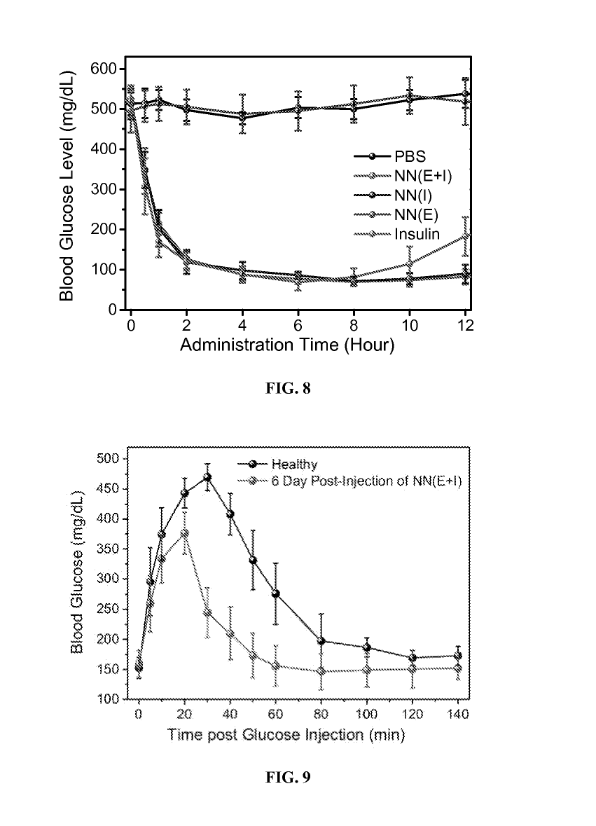

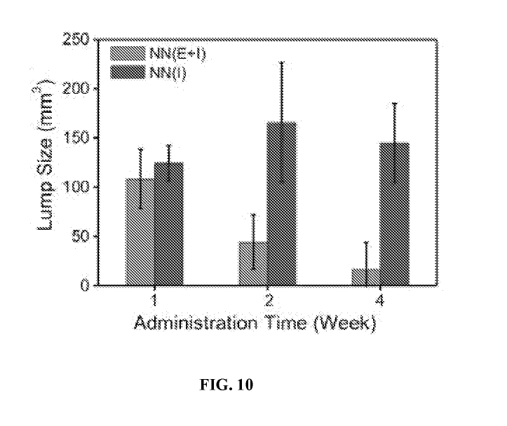

Insulin release kinetics in response to varying glucose levels were assessed. Accumulated insulin release studies were performed and confirmed that faster insulin release was achieved from the nano-networks under a hyperglycemic environment, as plotted in FIGS. 6A-6C. In contrast, limited insulin release from the nano-networks was observed within 15 hours of incubation at the normal glucose level and glucose-free PBS buffer. These results are also consistent with the dissociation response discussed above. Meanwhile, the solubility of insulin is increased under an acidic environment, which further enhances the insulin release rate.

The insulin release profile of nano-networks presented a pulsatile pattern when the glucose concentration was cyclically varied between the normal and hyperglycemic levels every 2 hours for several repetitions. The nano-network responded to changes of glucose levels and a maximum 3.6-fold increase in the insulin release rate was obtained when the glucose level was switched to the hyperglycemic state. Moreover, the release rates at both hyperglycemic and normoglycemic levels stably increased to a maximum point and then gradually decreased. The “acceleration period” of the release rate can be attributed to the progressively attenuated cohesive force and dissociated structure of the network. Collectively, these results point to the degradation of nano-network and the subsequent insulin release is a glucose-mediated and pH-dependent process. Like a smart valve system, the insulin release through nano-network is facilitated at a high glucose level and inhibited at a low glucose level.

### Example 4

### In Vivo Studies in STZ-Induced Diabetic Mice

Materials and Methods

The efficacy of the insulin-loaded nano-network for diabetes treatment was evaluated in vivo by assessment of glycemia in STZ-induced adult diabetic mice (male C57B6, Jackson Lab, USA). Mice were cared for under supervision of MIT's Division of Comparative Medicine and in compliance with NIT's Principles of Laboratory Animal Care.

The blood glucose levels of mice were continuously tested for two days before administration by collecting blood (˜3 μL) from the tail vein and measuring using the Clarity GL2Plus Glucose Monitor (VWR, USA). Eight diabetic mice were selected for each group administered with PBS solution, insulin solution, nano-network loaded with human recombinant insulin and enzymes, nano-network loaded with insulin only or nano-network with enzyme only. 150 μL of the aqueous solution or nano-network was injected using a 1 cc syringe with a 19-gauge needle into the subcutaneous dorsum of mice (insulin dose: 60 mg/kg) that had been anesthetized with 1% isoflurane. The glucose level of each mouse was monitored over time (every 30 min or 2 hours for the first 12 hours in the day of administration and once per day in the morning for following days).

To measure in vivo insulin concentration, blood samples (25 μL) were drawn from the tail vein of mice and collected into Sarstedt serum gel microtubes. Serum samples (5 μL) were stored frozen at −20° C. until assayed. Plasma insulin concentrations were determined using the human insulin ELISA kit (Calbiotech, USA). To quantitatively determine ratios of glycated albumin over total albumin concentration, collected serum samples (7 μL) were analyzed one day before administration and two weeks after administration using the mouse glycated albumin kit (Crystal Chem, USA).

To assess the biocompatibility of nano-network, the cytotoxicity of nanoparticles coated with chitosan or alginate and their degradation products toward HeLa cells was evaluated at various concentrations ranging from 0.05 mg/mL to 1.2 mg/mL.

Results

To examine the efficacy of the insulin-loaded nano-network for diabetes treatment, streptozotocin (STZ)-induced diabetic mice31 were subcutaneously injected with PBS solution, human recombinant insulin solution, nano-network loaded with insulin and enzymes (NN(E+I)), nano-network loaded with insulin only (NN(I)) and nano-network loaded with enzymes only (NN(E)). The blood glucose (BG) levels of administrated mice in each group were then monitored over time. The BG levels of diabetic mice treated with one injection of NN-EI were stably maintained in the normoglycemia (<200 mg/dL) range for up to 10 days without peaks of hyperglycemic or hypoglycemic states.

For some of the treated mice, the BG levels were maintained in the normoglycemic range for over two weeks. The average BG levels of the NN(E+I) group gradually increased due to a decrease in the insulin content or loss of bioactivity of encapsulated insulin, but were still significantly lower than the original BG level for up to three weeks. Correspondingly, the plasma human insulin in mice injected with NN(E+I) can be detected over the three week time course.

The rapid decrease in BG levels in the first 12 hours is likely due to the initial burst of the insulin released in the solution part or adhered on the surface of the nano-network. However, the subsequent release of the remaining insulin encapsulated in nanoparticles, mediated by enzymatic catalysis, took place more slowly. In contrast, the BG levels of mice treated with NN(I) maintained BG levels within the normal range for two days before returning to the hyperglycemic state. Meanwhile BG levels of mice injected with insulin solution increased to the hyperglycemic range on the second day after administration. In the absence of enzymes, NN(I) cannot undergo acidic-degradation event to effectively release insulin and thus the BG levels of administrated mice were notably higher than those treated with NN(E+I). Without a loading matrix, pure insulin was quickly cleared and resulted in a rapid decrease in plasma insulin concentration on the next day of administration.

To investigate the possible effect of the catalytic consumption of glucose on the decrease of the BG levels, diabetic mice were treated with NN(E). However, the conversion of glucose through enzymes did not show a detectable effect in lowering BG levels. The glucose-responsiveness was tested in vivo by an intravenous glucose tolerance test at 6 day post-injection. Mice treated with NN(E+I) showed a rapid increase in BG levels upon glucose injection followed by a slightly delayed decrease to finally reach normal BG levels at 80 min in comparison with healthy animals. The glycated albumin level in serum, a medium term (2-3 weeks) indicator of diabetic control, was also tested. The glycated albumin ratio (glycated albumin/total albumin) was prominently decreased 1.6-fold two weeks after administration with NN(E+I). In addition, after a 4-week post-administration period, the group treated with NN(E+I) gained higher body condition scores compared with control groups. Taken together, sustained insulin release from the degradable insulin-loaded nano-network and the pharmacological duration of insulin activity have been observed.

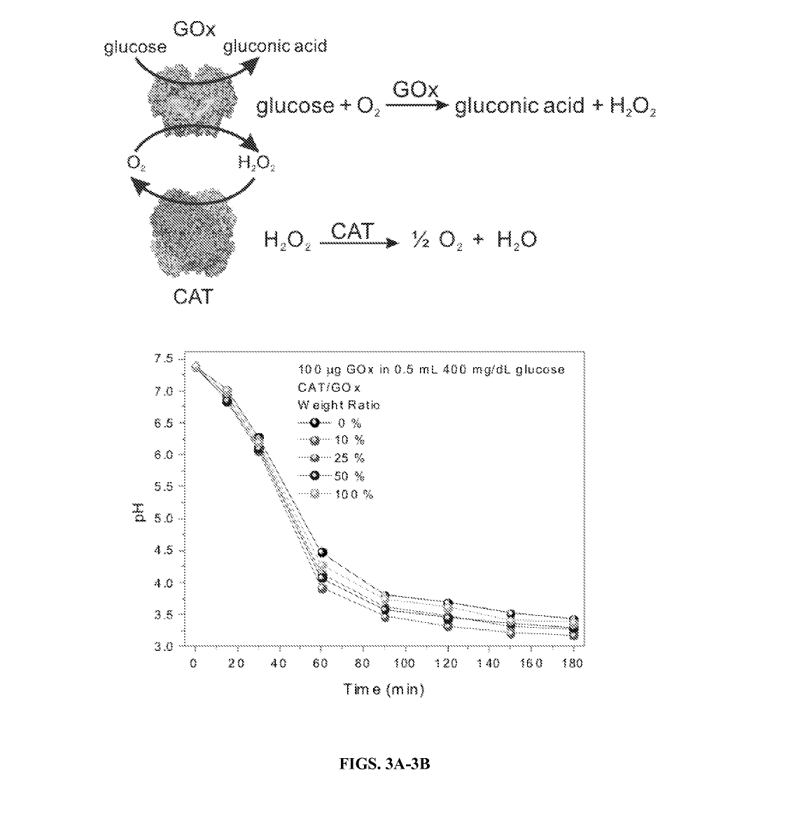

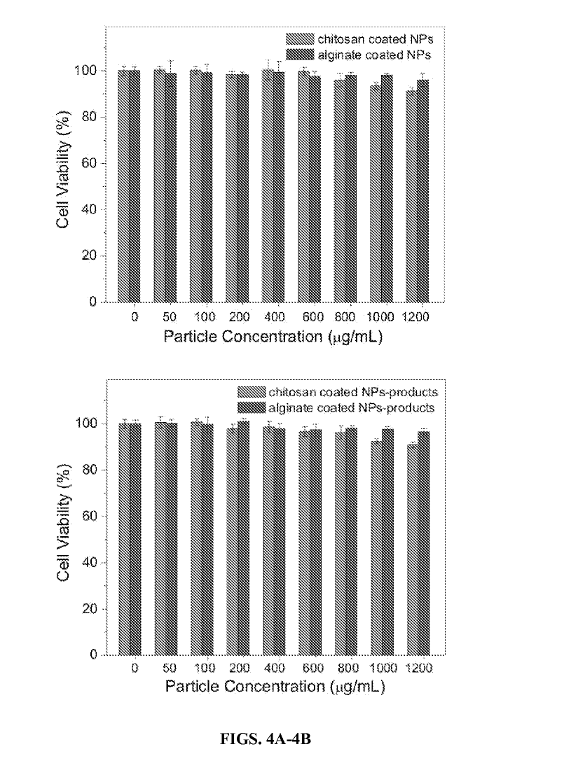

For all concentrations studied, m-dextran based nanoparticles and relevant degradation products did not show significant decrease of cell viability (FIGS. 4A and 4B). To further investigate in vivo biocompatibility and degradability of nano-network, the sizes of skin protrusion due to the subcutaneous injection were monitored over time. As shown in FIG. 10, the averaged lump sizes in the injection sites of mice treated with NN(E+I) steadily decreased, suggesting glucose-mediated degradation was substantially triggered. No significant skin protrusion can be found after 4 weeks. However, for mice treated with NN(I), the lump sizes did not obviously decrease, even after 4 weeks. The histological response of the injected nano-network shows that during the first 2 weeks, inflammatory cells, such as polymorphnuclear cells (PMN) and macrophages, infiltrated into both NN(E+I) and NN(I), where the nano-network region was avascular and acellular, and a fibrin network separated the nano-network from the muscle region. This inflammatory reaction can be attributed to the acute inflammation which often occurs when biomaterials are subcutaneously implanted. After 4 weeks, the NN(E+I) had completely degraded and the affected region was covered with connective tissue. However, a noticeable inflammatory region can still be observed in the samples administrated with NN(I).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. An injectable polymeric nanoparticle-crosslinked network formulation for controlled release of a therapeutic, prophylactic or diagnostic agent, comprising particles comprising:
an acid-degradable polymer matrix,
a responsive signaling component, and
a therapeutic, prophylactic or diagnostic agent,
wherein a first plurality of the particles have a first nonzero charge on the surface and a second plurality of the particles have a second opposite nonzero charge on the surface, which interact to form an injectable polymeric nanoparticle-crosslinked network,
wherein the responsive signaling component generates acid in the presence of a physiological component, wherein the acid degrades the polymer to release the therapeutic, prophylactic or diagnostic agent.

2. The formulation of claim 1 wherein the agent is insulin or insulin analog or an agent that increases insulin concentration.

3. The formulation of claim 2, wherein the responsive signaling component comprises glucose oxidase and catalase.

4. The formulation of claim 3, wherein the ratio (w/w) of glucose oxidase and catalase to acid-degradable polymer matrix is from 1:100 to 1:15.

5. The formulation of claim 4, wherein the glucose oxidase and catalase are present in a ratio (w/w) of 4:1.

6. The formulation of claim 1, wherein the first plurality of particles having a positive charge on their surface and the second plurality of particles having a negative charge on their surface interact to form a gel.

7. The formulation of claim 6, wherein the zeta potential of the particles in the first plurality of particles and the zeta potential of the particles in the second plurality of particles have a magnitude from 5 to 15 mV.

8. The formulation of claim 6, wherein the particles in the first plurality of particles further comprise a surface modifier.

9. The formulation of claim 8, wherein the surface modifier is chitosan or alginate.

10. The formulation of claim 1, wherein the particles have a hydrodynamic radius of less than 350 nm.

11. The formulation of claim 1, wherein the acid-degradable polymer matrix comprises a cross-linkable polymer and an acid-degradable cross linker.

12. The formulation of claim 1, wherein the acid-degradable polymer matrix comprises a polymer having a plurality of hydrolysable moieties.

13. The formulation of claim 2, wherein the formulation dissociates under hyperglycemic conditions and does not substantially dissociate in normal glucose levels.

14. The formulation of claim 13, wherein the formulation dissociates after 8 hours in a glucose concentration of 400 mg/dL.

15. The formulation of claim 13, wherein the formulation does not substantially dissociate after 15 hours in normal glucose levels.

16. The formulation of claim 13, wherein the release of the insulin or insulin analog or agent that increases insulin concentration is pulsatile when glucose concentrations are cyclically varied between normal and hyperglycemic conditions.

17. A method of treating a patient in need thereof comprising administering to an individual in need thereof an effective amount of the formulation of claim 1.

18. The method of claim 17 wherein the agent is insulin or insulin analog or agent that increases insulin concentration and the individual has type 1 or type 2 diabetes.

19. The method of claim 18 comprising administering the formulation to maintain normoglycemia, normal glycalated albumin levels, or higher body condition score.

20. The method of claim 19 administered in an effective amount to maintain blood glucose concentrations at a between 70-130 mg/dL or 90-110 mg/dL.
